(19) United States
(12) Patent Application Publication
(10) Pub. No.: US 2024/0165150 A1
(43) Pub. Date: May 23, 2024

(54) COMBINATION FOR TREATMENT OF THROMBOINFLAMMATION

(71) Applicant: TX MEDIC AB, Viken (SE)

(72) Inventor: Adam BRUCE, Viken (SE)

(21) Appl. No.: 18/551,936

(22) PCT Filed: Mar. 21, 2022
(86) PCT No.: PCT/SE2022/050264

§ 371 Date: Sep. 22, 2023

(30) Foreign Application Priority Data
Mar. 24, 2021 (SE) 2150338-8

(51) Int. Cl. A61K 31/737; A61K 31/727; A61P 37/06

(52) U.S. Cl. CPC A61K 31/737; A61K 31/727; A61P 37/06

(57) ABSTRACT

The invention relates to a combination comprising an effective amount of dextran sulfate, or a pharmaceutically acceptable salt thereof, and an effective amount of heparin, or a pharmaceutically acceptable salt thereof, for use in treatment of thromboinflammation, such as instant blood-mediated inflammatory reaction (IBMIR) and ischemia-reperfusion injury (IRI). The invention is based on the surprising finding that heparin, or the pharmaceutically acceptable salt thereof, can synergistically boost anti-thromboinflammatory effects of dextran sulfate, or the pharmaceutically acceptable salt thereof.

### TECHNICAL FIELD

The present invention generally relates to a combination of dextran sulfate and heparin, and in particular to the use of such a combination of dextran sulfate and heparin in treatment of thromboinflammation.

### BACKGROUND

Thromboinflammation, also referred to as thromboinflammatory reaction, is an inflammatory reaction mediated by humoral as well as cellular components of the innate immune system, which results in thrombosis, inflammation and cell dysfunction and cell death.

Thromboinflammation occurs upon exposure of a biomaterial to blood in a host subject, in connection with cell transplants and during ischemia-reperfusion injury (IRI). Once a biomaterial, such as of an implantable medical device or catheter, makes contact with the blood, it is recognized as foreign by the innate immune system, which responds by upregulating a variety of cascade systems to cause activation of platelets, deposition of coagulation and complement proteins, and generation of inflammatory cytokines, with the end result being development of thromboinflammation.

Instant blood-mediated reaction (IBMIR) is a thromoboinflammatory response of the innate immune system triggered by the exposure or contact of cells or cell clusters with foreign blood. IBMIR is characterized by expression of tissue factor (TF) on the cells, which triggers a local generation of thrombin. Subsequently, activated platelets adhere to the cell surface promoting activation of both the coagulation and complement systems. In addition, white blood cells are recruited and infiltrate the cells. These effects together cause a disruption and destruction of the cell morphology within the first few hours after contact with the foreign blood. IBMIR also accelerates the subsequent cell-mediated specific immune response in a later phase.

IRI, also referred to as reperfusion injury or reoxygenation injury, is the tissue damage caused when blood supply returns to tissue after a period of ischemia or lack of oxygen (anoxia or hypoxia). The absence of oxygen and nutrients from blood during the ischemic period creates a condition, in which the restoration of circulation results in inflammation and oxidative damage through the induction of oxidative stress rather than restoration of normal function. Ischemia and hypoxia disrupts the regulatory mechanisms of the endothelium and results in the loss of vascular protective barriers, in particular the vascular glycocalyx, and in an increase in vascular permeability. The hypoxic endothelium not only expresses TF and proinflammatory cytokines and chemokines but also deposits complement on the endothelial surface, leading to the triggering of a local inflammation and the binding of platelets and infiltration of leukocytes. This response leads to a further loss of integrity of the endothelial cells, ultimately causing vascular damage. When blood circulation returns, i.e., reperfusion, the hypoxic cells are attacked by the innate immune system, which recognizes the cells as “altered self.” This attack further aggravates the condition and finally leads to cell death.

Low-molecular weight dextran sulfate (LMW-DS) has been shown to be effective in treatment of thromboinflammation, such as IBMIR (U.S. Pat. Nos. 8,629,123; 8,901,104; 8,906,884; 9,364,499). Another sulfated polysaccharide, heparin, has also been tested for use in treatment of thromboinflammation. However, LMW-DS has been shown to outperform heparin (Cell transplantation 26(1): 71-81 (2017); Gustafson, Thromboinflammation in a Model of Hepatocyte Transplantation, Digital Comprehensive Summaries of Uppsala Dissertations from the Faculty of Medicine 123. 75 pp. (2016), Uppsala: Acta Universitatis Upsaliensis. ISBN 978-91-554-9592-3).

### SUMMARY

It is a general objective to provide a treatment of thromboinflammation.

It is another objective to provide an IBMIR model that can be used to analyze the effectiveness of treatment of IBMIR.

These and other objectives are met by embodiments as disclosed herein.

The present invention is defined in the independent claims. Further embodiments of the invention are defined by the dependent claims.

An aspect of the invention relates to a combination comprising an effective amount of dextran sulfate, or a pharmaceutically acceptable salt thereof, and an effective amount of heparin, or a pharmaceutically acceptable salt thereof, for use in treatment of thromboinflammation.

Another aspect of the invention relates to a transplant composition comprising a transplant, dextran sulfate, or a pharmaceutically acceptable salt thereof, and heparin, or a pharmaceutically acceptable salt thereof.

A further aspect of the invention relates to an in vitro IBMIR model method. The method comprises adding in vitro test cells to a blood loop comprising whole blood to trigger IBMIR, adding in vitro dextran sulfate, or a pharmaceutically acceptable salt thereof, to the blood loop, and adding in vitro heparin, or a pharmaceutically acceptable salt thereof, to the whole blood in the blood loop. The method also comprises determining an effectiveness of a combination of dextran sulfate, or the pharmaceutically acceptable salt thereof, and heparin, or the pharmaceutically acceptable salt thereof, to inhibit IBMIR by determining at least one hematological parameter, at least one coagulation parameter, at least one complement parameter and at least one platelet parameter.

Yet another aspect of the invention relates to an in vitro IBMIR model kit. The kit comprises a tubing and a connector configured to interconnect the tubing to form a tubing loop. The kit also comprises dextran sulfate, or a pharmaceutically acceptable salt thereof and heparin, or a pharmaceutically acceptable salt thereof. The heparin, or the pharmaceutically salt thereof, is not immobilized to an inner surface of the tubing or the connector.

The invention is based on the surprising finding that heparin, or the pharmaceutically acceptable salt thereof, can synergistically boost anti-thromboinflammatory effects of dextran sulfate, or the pharmaceutically acceptable salt thereof. In more detail, heparin, or the pharmaceutically acceptable salt thereof, alone had no effect on platelet binding to cell transplants and to monocytes or granulocytes. However, the effects of dextran sulfate, or the pharmaceutically acceptable salt thereof, in reducing such platelet binding were significantly improved when combined with heparin, or the pharmaceutically acceptable salt thereof. Furthermore, heparin, or the pharmaceutically acceptable salt thereof, alone did not have any effect on complement activation on platelets. However, heparin, or the pharmaceutically acceptable salt thereof, was capable of enhancing the inhibitory effect of dextran sulfate, or the pharmaceutically acceptable salt thereof, on such complement activation on platelets.

### BRIEF DESCRIPTION OF THE DRAWINGS

The embodiments, together with further objects and advantages thereof, may best be understood by making reference to the following description taken together with the accompanying drawings, in which:

FIGS. 1A to 1C illustrate platelet (PLT) counts at zero and 1 hour time points.

FIGS. 2A to 2C illustrate red blood cell (RBC) counts at zero and 1 hour time points.

FIGS. 3A to 3C illustrate white blood cell (WBC) counts at zero and 1 hour time points.

FIGS. 4A to 4C illustrate neutrophil counts and total WBC counts at zero and 1 hour time points.

FIGS. 5A to 5C illustrate lymphocyte counts and total WBC counts at zero and 1 hour time points.

FIGS. 6A to 6C illustrate monocyte counts and total WBC counts at zero and 1 hour time points.

FIGS. 7A to 7C illustrate basophil counts and total WBC counts at zero and 1 hour time points.

FIGS. 8A to 8C illustrate eosinophil counts and total WBC counts at zero and 1 hour time points.

FIGS. 9A to 9E illustrate cytokines analyzed in blood extracted from loops at zero and 4 hour time 15 points: (A) IL-6, (B) IL-8, (C) TNF-α, (D) IP-10 and (E) MCP1.

FIGS. 10A and 10B illustrate the proportion of viable, apoptotic and dead WI-38 cells (A) and tissue factor positive (TF+) cells (B). 20 FIGS. 11A and 11B illustrates frequency and viability of TF+ cells at zero and 1 hour time points (A) and proportion of C5b-9 positive TF+ cells (B).

FIGS. 12A and 12B illustrate frequency of apoptotic (A) and dead (B) TF+ cells at zero and 1 hour time points.

FIGS. 13A and 13B illustrate frequency (A) and activation of granulocytes (CD66b+) (B) at the zero and 1 hour time points.

FIGS. 14A and 14B illustrate frequency (A) and activation monocytes (CD14+) (B) at the zero and 1 hour 30 time points.

FIGS. 15A and 15B illustrate frequency (A) and activation B cells (CD19+) (B) at the zero and 1 hour time points.

FIGS. 16A and 16B illustrate frequency (A) and activation T cells (CD3+) (B) at the zero 1 hour time points.

FIG. 17 illustrates frequency of platelets at the zero and 1 hour time points.

FIG. 18 illustrates activation of platelets at the 1 hour time point compared to zero time point.

FIGS. 19A and 19B illustrate binding of platelets to monocytes at the 1 hour time point (A) and proportion of activated (CD62P+) platelets of total monocyte-bound platelets (B).

FIGS. 20A and 20B illustrate binding of platelets to granulocytes at the 1 hour time point (A) and proportion of activated (CD62P+) platelets of total granulocyte-bound platelets (B).

FIGS. 21A and 21B illustrate binding of platelets to WI-38 cells detected by fibroblast marker (A) and formation of C5b-9 complex on platelets (B) at the zero and 1 hour time points.

FIGS. 22A and 22B illustrate complement split product C3a concentrations measured by ELISA at 15 min (A) and 1 hour (B) time points compared to zero time point. Dashed line=lower limit of quantification (LLOQ).

FIGS. 23A and 23B illustrate complement split product C5a concentrations measured by ELISA at 15 min (A) and 1 hour (B) time points compared to zero time point. Dashed line=LLOQ.

### DETAILED DESCRIPTION

The present invention generally relates to a combination of dextran sulfate and heparin, and in particular to the use of such a combination of dextran sulfate and heparin in treatment of thromboinflammation.

Allogeneic and xenogeneic cells and cell transplants elicit IBMIR when exposed to the blood of a recipient patient. As a consequence, within a few hours after transplantation the morphology of the cells become disrupted and destroyed, generally manifested in loss of integrity, structure and form of the cells.

Administrations of conventional immunosuppressive drugs that prevent production of antibodies and organ-rejection have no effect on IBMIR or the graft-rejection of cell transplants caused by IBMIR. This indicates that the main mechanisms of IBMIR and graft-rejection of cell transplants differs from the rejection mechanism found in transplantation of whole organs and vascularized tissue.

Hereinafter follows a more detailed survey of the symptoms of IBMIR, and in particular platelet consumption, coagulation and complement activation and white blood cell infiltration.

Starting with platelet consumption, IBMIR affects the platelet count of blood exposed to allogeneic or xenogeneic cells or cell clusters. A significant decrease in free circulating platelets can be detected in the blood following the blood-cell contact. The platelets become activated and adhere to the cells, resulting in a platelet aggregation. Following adhesion to the cells, the platelets release several substances including platelet phospholipids, which are important for clot formation and activation of the coagulation system.

Upon contact with blood, the foreign cells activate the coagulation system, through the expression of tissue factors on the cells and through substances released by the adhering and aggregating platelets. Briefly, tissue factor (TF) produced by the cells complexes with blood coagulation factor VIIa (FVIIa) and acts enzymatically on factor X (FX) to form activated factor X (FXa). Thereafter follows a cascade of activation of different factors, which eventually results in generation of thrombin from prothrombin. Thrombin in turn causes polymerization of fibrinogen molecules into fibrin fibers forming a fibrin clot around the cells, which is all well known to a person skilled in the art. Thrombin also activates the intrinsic pathway for initiating blood clotting, in which factor XII (FXII, Hageman factor) becomes activated (FXIIa) and in turn enzymatically activates factor XI (FXI, thromobplastin antecedent), resulting in FXIa, the activated form of factor XI. Also this pathway eventually results in generation of thrombin from prothrombin as for the extrinsic TF-activated pathway.

The blood clotting may be inhibited by antithrombin, a circulating serine protease inhibitor, which inactivates FXIIa, FXIa and thrombin, forming factor XIIa-antithrombin (FXIIa-AT), factor XIa-antithrombin (FXIa-AT) and thrombin-antithrombin (TAT) complexes. In addition, C1 esterase inhibitor is a known inhibitor of FXIa and FXIIa forming factor XIa-C1 esterase inhibitor (FXIa-C1 INH) and factor XIIa-C1 esterase inhibitor (FXIIa-C1 INH) complexes.

A once formed fibrin clot around the cells or cell clusters may be removed by the action of plasmin of the fibrinolytic system. Plasmin degrades the fibrin clot into fibrin degradation products, thereby preventing further clotting. However, the action of plasmin is inhibited by alpha 2 antiplasmin, which binds to and inactivates free plasmin forming a plasmin-alpha 2 antiplasmin (PAP) complex.

IBMIR is characterized by formation of fibrin clots around cells exposed to foreign blood in vitro and in vivo.

Following platelet and coagulation activation, a complement cascade takes place in IBMIR. One of the components of the complement system is C3, which, when activated, is cleaved into the small C3a fragment, a peptide mediator of inflammation, and the larger fragment C3b. C3b in turn binds to other components of the complement system forming C5 convertase, which cleaves C5 into C5a, which diffuses away, and the active form C5b, which attaches to the cell surface. The bound C5b then binds to four more complement components forming the membrane attack complex c5b-9. This complex displaces the membrane phospholipids forming large transmembrane channels, which disrupts the membrane and enables ions and small molecules to diffuse freely. Thus, the cell cannot maintain its osmotic stability and is lysed by an influx of water and loss of electrolytes.

Most of the platelet consumption has already occurred before the complement mediated effects of IBMIR can be detected, suggesting that the clotting reaction may induce complement activation. IBMIR causes significant complement activation as measured by an increase of C3a and C5a in the blood and C5b-9 positive platelets.

IBMIR is also characterized by infiltration of white blood cells (WBCs) into the cells or cell clusters. Infiltration of CD11b+ polymorphonuclear cells and monocytes into the cells is clearly detected by immunohistochemical staining. Hence, IBMIR is characterized by a reduction in WBC count, and in particular in neutrophil counts, monocyte counts and eosinophil counts.

Dextran sulfate has been shown to be effective in treatment of IBMIR. In particular, dextran sulfate causes a reduction of platelets adhering to the cell transplant. Dextran sulfate also abrogates the effects of IBMIR on coagulation activation, which is manifested in a decrease in the amount of FXIa-AT, FXIIa-AT, TAT and PAP detected in the blood. As a result, dextran sulfate reduces blood clotting and reduces the risk of hemolysis following contact between the cell transplant and the recipient patient's blood. Dextran sulfate further reduces the amount of complement components in the blood, in particular C3a and C5a. In addition, dextran sulfate reduces formation of the C5b-9 complex on platelets.

Hematological analyses showed that dextran sulfate was capable of restoring WBC counts, including normalizing neutrophil, monocyte and eosinophil counts.

Heparin has also been proposed for the treatment of IBMIR. However, dextran sulfate has been shown to be far superior as compared to heparin in treating IBMIR. For instance, dextran sulfate is significantly more efficient than heparin in inhibiting the generation of TAT and FXIIa/AT and in suppressing the activation of the complement system. In fact, no significant effect at all was observed with heparin in terms of preventing the activation of the complement system. Furthermore, heparin does not affect WBC counts, including neutrophil, monocyte and eosinophil counts, nor does heparin reduce platelets adhering to the cell transplant or formation of the C5b-9 complex on platelets.

Ischemia-reperfusion injury (IRI) is the tissue damage caused when blood supply returns to tissue after a period of ischemia or lack of oxygen (anoxia or hypoxia). The absence of oxygen and nutrients from blood during the ischemic period creates a condition, in which the restoration of circulation results in inflammation and oxidative damage through the induction of oxidative stress rather than restoration of normal function. Ischemia and hypoxia disrupts the regulatory mechanisms of the endothelium and results in the loss of vascular protective barriers, in particular the vascular glycocalyx, and in an increase in vascular permeability. The hypoxic endothelium not only expresses TF and proinflammatory cytokines and chemokines but also deposits complement on the endothelial surface, leading to the triggering of a local inflammation and the binding of platelets and infiltration of leukocytes. This response leads to a further loss of integrity of the endothelial cells, ultimately causing vascular damage. When blood circulation returns, i.e., reperfusion, the hypoxic cells are attacked by the innate immune system, which recognizes the cells as “altered self.” This attack further aggravates the condition and finally leads to cell death. Reperfusion of ischemic tissues is often associated with microvascular injury, particularly due to increased permeability of capillaries and arterioles that lead to an increase of diffusion and fluid filtration across the tissues. Activated endothelial cells produce more reactive oxygen species (ROS) but less nitric oxide following reperfusion, and the imbalance results in a subsequent inflammatory response. The inflammatory response is partially responsible for the damage of reperfusion injury. White blood cells, carried to the area by the newly returning blood, release a host of inflammatory factors, such as interleukins as well as free radicals in response to tissue damage. The restored blood flow reintroduces oxygen within cells that damages cellular proteins, DNA, and the plasma membrane. Damage to the cell's membrane may in turn cause the release of more free radicals. Such reactive species may also act indirectly in redox signaling to turn on apoptosis. White blood cells may also bind to the endothelium of small capillaries, obstructing them and leading to more ischemia.

IRI is of a main concern in a number of diseases and conditions including stroke, myocardial infarction and (solid) organ and tissue transplantation. In the latter case, IRI significantly influences short-term as well as long-term transplantation outcomes. Clinically, IRI is associated with delayed function and both acute and chronic rejection of the transplanted organ or tissue. Repeated bouts of ischemia and reperfusion injury are also thought to be a factor leading to the formation and failure to heal of chronic wounds, such as pressure sores and diabetic foot ulcer. Continuous pressure limits blood supply and causes ischemia, and the inflammation occurs during reperfusion. As this process is repeated, it eventually damages tissue enough to cause a wound.

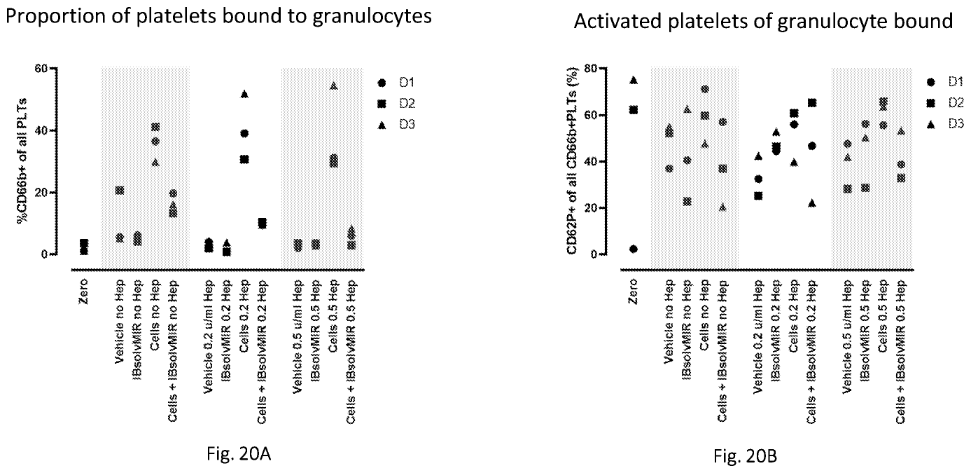

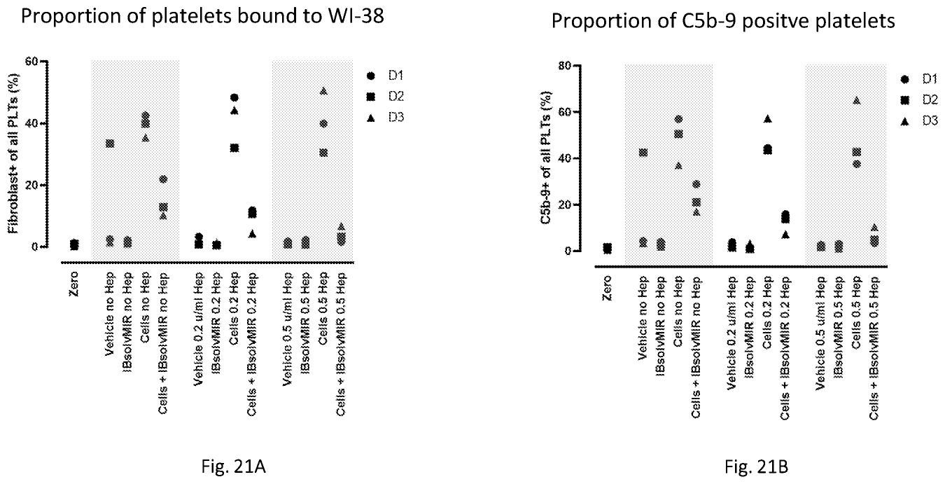

The present invention is based on the surprising finding that heparin, when combined with dextran sulfate, is capable of synergistically boosting beneficial effects of dextran sulfate against thromboinflammation, such as IBMIR or IRI. In more detail, administration of heparin alone did not have any effect at all on the proportion of platelets that are bound to monocytes (FIG. 19A), bound to granulocytes (FIG. 20A), or bound to the cell transplant (FIG. 21A). Furthermore, administration of heparin alone did not have any effects on the proportion of platelets that are C5b-9 positive (FIG. 21B).

Dextran sulfate alone was capable of reducing the proportions of platelets that are bound to monocytes (FIG. 19A), bound to granulocytes (FIG. 20A), or bound to the cell transplant (FIG. 21A) and capable of reducing the proportion of C5b-9 positive platelets (FIG. 21B). However, when combining dextran sulfate with heparin, which alone did not have any effects on any of these parameters of thromboinrflammation, heparin was capable of boosting the positive effects of dextran sulfate. This boosting effect of heparin was highly surprising since heparin alone did not have any effects on the proportions of platelets bound to monocytes, granulocytes or to the cell transplant, or the proportion of C5b-9 positive platelets. Hence, it would therefore be expected that combining dextran sulfate with heparin would not lead to any changes in these thromboinflammatory parameters beyond what is seen from administration of dextran sulfate. This means that the effects obtained when combining dextran sulfate with heparin are beyond the combined effects as seen when administering each of dextran sulfate and heparin alone. Accordingly, a true synergistic effect is achieved by combining dextran sulfate and heparin.

The expression “cell transplant” generally refers, in the present invention, to a single cell, several single cells or a cluster of many cells to be transplanted into a recipient body of, preferably, a mammalian, and especially, a human patient. Also larger cell clusters of non-vascularized tissues are comprised in the expression cell transplant, as used herein. Cell transplants also include membrane-bound extracellular vesicles, such as exosomes and ectosomes.

An illustrative, but non-limiting, example of cell transplant according to the present invention are allogeneic or xenogeneic islets of Langerhans transplanted into the portal vein of the liver of patients suffering from type I diabetes. Further examples of cell transplants include hepatocytes, fibroblasts, stem cells, including mesenchymal stem cells (MSCs), also known as stromal stem cells in the art, and other cells expressing tissue factor (TF) or are induced to express TF when in contact with the blood of the recipient body.

An aspect of the invention relates to a combination comprising an effective amount of dextran sulfate, or a pharmaceutically acceptable salt thereof, and an effective amount of heparin, or a pharmaceutically acceptable salt thereof, for use in treatment of thromboinflammation.

Dextran sulfate is a sulfated polysaccharide and in particular a sulfated glucan, i.e., a polysaccharide made of many glucose molecules. Correspondingly, heparin is sulfated glycosaminoglycan, i.e., a polysaccharide of repeating disaccharide units, in particular 1,4-linked disaccharide repeating units of uronic acid and glucosamine residues. Average molecular weight as defined herein indicates that individual polysaccharides or glycosaminoglycans may have a molecular weight different from this average molecular weight but that the average molecular weight represents the mean molecular weight of the polysaccharides or glycosaminoglycans. This further implies that there will be a natural distribution of molecular weights around this average molecular weight for a dextran sulfate sample and for a heparin sample.

Dextran sulfate is preferably a so-called low molecular weight dextran sulfate.

In the following, reference to (average) molecular weight and sulfur content of dextran sulfate applies also to any pharmaceutically acceptable salt of dextran sulfate. Hence, the pharmaceutically acceptable salt of dextran sulfate preferably has the average molecular weight and sulfur content as discussed in the following embodiments.

Average molecular weight (M_{w}) of dextran sulfate is typically determined using indirect methods, such as gel exclusion/penetration chromatography, light scattering or viscosity. Determination of average molecular weight using such indirect methods will depend on a number of factors, including choice of column and eluent, flow rate, calibration procedures, etc.

Average molecular weight (M_{w}): ΣMᵢ³Nᵢ/ΣMᵢNᵢ, typical for methods sensitive to molecular size rather than numerical value, e.g., light scattering and size exclusion chromatography (SEC) methods. If a normal distribution is assumed, then a same weight on each side of M_{w}, i.e., the total weight of dextran sulfate molecules in the sample having a molecular weight below M_{w} is equal to the total weight of dextran sulfate molecules in the sample having a molecular weight above M_{w}.

In an embodiment, dextran sulfate, or the pharmaceutically acceptable salt thereof, preferably has an average molecular weight equal to or below 40 000 Da, more preferably equal to or below 20 000 Da and in particular equal to or below 10 000 Da.

Dextran sulfate of an average molecular weight exceeding 10 000 Da generally has a lower effect vs. toxicity profile as compared to dextran sulfate having a lower average molecular weight. This means that the maximum dose of dextran sulfate that can be safely administered to a subject is lower for larger dextran sulfate molecules (>10 000 Da) as compared to dextran sulfate molecules having an average molecular weight within the preferred range. As a consequence, such larger dextran sulfate molecules are less appropriate in clinical uses when the dextran sulfate is to be administered to subjects in vivo.

In an embodiment, dextran sulfate, or the pharmaceutically acceptable salt thereof, has an average molecular weight within a range of from 2 000 to 10 000 Da. In another embodiment, the average molecular weight is within a range of from 2 500 to 10 000 Da. In a particular preferred embodiment, the average molecular weight is within a range of from 3 000 to 10 000 Da.

In an optional, but preferred embodiment, less than 40% of the dextran sulfate molecules have a molecular weight below 3 000 Da, preferably less than 35%, such as less than 30% or less than 25% of the dextran sulfate molecules have a molecular weight below 3 000 Da. In addition, or alternatively, less than 20% of the dextran sulfate molecules have a molecular weight above 10 000 Da, preferably less than 15%, such as less than 10% or less than 5% of the dextran sulfate molecules have a molecular weight above 10 000 Da. Thus, in a particular embodiment, the dextran sulfate has a substantially narrow molecular weight distribution around the average molecular weight.

In a particular embodiment, the average molecular weight of dextran sulfate, or the pharmaceutically acceptable salt thereof, is within a range of from 3 500 to 9 500 Da, such as within a range of from 3 500 to 8 000 Da.

In another particular embodiment, the average molecular weight of dextran sulfate, or the pharmaceutically acceptable salt thereof, is within a range of from 4 500 to 7 500 Da.

In a further particular embodiment, the average molecular weight of dextran sulfate, or the pharmaceutically acceptable salt thereof, is within a range of from 4 500 to 5 500 Da.

Thus, in a currently preferred embodiment the average molecular weight of dextran sulfate, or the pharmaceutically acceptable salt thereof, is preferably approximately 5 000 Da or at least substantially close to 5 000 Da, such as 5 000±500 Da, for instance 5 000±400 Da, preferably 5 000±300 Da or 5 000±200 Da, such as 5 000±100 Da. Hence, in an embodiment, the average molecular weight of dextran sulfate, or the pharmaceutically acceptable salt thereof, is 4.5 kDa, 4.6 kDa, 4.7 kDa, 4.8 kDa, 4.9 kDa, 5.0 kDa, 5.1 kDa, 5.2 kDa, 5.3 kDa, 5.4 kDa or 5.5 kDa.

In a particular embodiment, the average molecular weight of dextran sulfate, or the pharmaceutically salt thereof, as presented above is average M_{w}, and preferably determined by gel exclusion/penetration chromatography, size exclusion chromatography, light scattering or viscosity-based methods.

Dextran sulfate is a polyanionic derivate of dextran and contains sulfur. The average sulfur content for dextran sulfate of the embodiments is preferably from 15 to 20% and more preferably approximately 17%, generally corresponding to about or at least two sulfate groups per glucosyl residue. In a particular embodiment, the sulfur content of dextran sulfate is preferably equal to or at least close to the maximum possible degree of sulfur content of the corresponding dextran molecules.

In a particular embodiment, dextran sulfate of the embodiments has a number average molecular weight (Mₙ) as measured by nuclear magnetic resonance (NMR) spectroscopy within a range of from 1850 to 3500 Da.

Number average molecular weight (Mₙ): ΣMᵢNᵢ/ΣNᵢ, typically derived by end group assays, e.g., NMR spectroscopy or chromatography. If a normal distribution is assumed, then a same number of dextran sulfate molecules can be found on each side of Mₙ, i.e., the number of dextran sulfate molecules in the sample having a molecular weight below Mₙ is equal to the number of dextran sulfate molecules in the sample having a molecular weight above Mₙ.

In a preferred embodiment, dextran sulfate of the embodiments has a Mₙ as measured by NMR spectroscopy within a range of from 1850 to 2500 Da, preferably within a range of from 1850 to 2300 Da, and more preferably within a range of from 1850 to 2000 Da.

In a particular embodiment, dextran sulfate of the embodiments has an average sulfate number per glucose unit within a range of from 2.5 to 3.0, preferably within a range of from 2.5 to 2.8, and more preferably within a range of from 2.6 to 2.7.

In a particular embodiment, dextran sulfate of the embodiments has an average number of glucose units within a range of from 4.0 to 6.0, preferably within a range of from 4.5 to 5.5, and more preferably within a range of from 5.0 to 5.2, such as about 5.1.

In another particular embodiment, dextran sulfate of the embodiments has on average 5.1 glucose units and an average sulfate number per glucose unit of 2.6 to 2.7, typically resulting in a number average molecular weight (Mₙ) as measured by NMR spectroscopy within a range of from 1850 to 2000 Da.

A dextran sulfate, or pharmaceutically salt thereof, that can be used according to the embodiments is described in WO 2016/076780.

In the following, reference to (average) molecular weight of heparin applies also to any pharmaceutically acceptable salt of heparin. Hence, the pharmaceutically acceptable salt of heparin preferably has the average molecular weight as discussed in the following embodiments.

In an embodiment, the heparin, or the pharmaceutically acceptable salt thereof, has an average molecular weight equal to or below 40 000 Da.

In a particular embodiment, the heparin, or the pharmaceutically acceptable salt thereof, has an average molecular weight equal to or below 30 000 Da, preferably equal to or smaller than 20 000 Da, and more preferably equal to or smaller than 17 500 Da.

In an embodiment, the heparin, or the pharmaceutically acceptable salt thereof, is unfractionated heparin (UFH), or a pharmaceutically acceptable salt thereof, comprising anioinic, sulfated glycosaminoglycan polymers with weights within an interval of from 1 000 Da to 40 000 Da. In a particular embodiment, the UFH, or the pharmaceutically acceptable salt thereof, comprises anioinic, sulfated glycosaminoglycan polymers with weights within an interval of from 2 000 Da to 35 000 Da, and preferably within an interval of from 3 000 and 30 000 Da.

In an embodiment, the UFH, or a pharmaceutically acceptable salt thereof, has an average molecular weight selected within an interval of from 10 000 Da to 20 000 Da, preferably within an interval of from 10 000 Da to 17 500 Da, and more preferably within an interval of from 12 000 Da to 15 000 Da.

UFH is a naturally occurring anticoagulant released from mast cells. UFH binds reversibly to antithrombin III (ATIII) and greatly accelerates the rate at which ATIII inactivates the coagulation enzymes thrombin (factor IIA) and factor Xa.

In another embodiment, the heparin, or the pharmaceutically acceptable salt thereof, is a low-molecular weight heparin (LMWH), or a pharmaceutically acceptable salt thereof. In such a case, the LMWH, or the pharmaceutically acceptable salt thereof, preferably has an average molecular weight equal to or below 10 000 Da. In a particular embodiment, the LMWH, or the pharmaceutically acceptable salt thereof, has an average molecular weight within a range of from 2 000 to 10 000 Da, preferably within a range of from 3 000 to 10 000 Da, more preferably within a range of from 3 500 to 9 500 Da, and most preferably within a range of from 3 500 to 5 500 Da.

The dextran sulfate according to the embodiments can be provided as a pharmaceutically acceptable salt of dextran sulfate. Pharmaceutically acceptable salt of dextran sulfate refers to a salt of dextran sulfate having the effects as disclosed herein and not being deleterious to the recipient thereof at the administered dose(s). Such pharmaceutically acceptable salts include e.g., a sodium or potassium salt of dextran sulfate. In a particular embodiment, the pharmaceutically acceptable salt is a sodium salt of dextran sulfate.

In a particular embodiment, the sodium salt of dextran sulfate, including Na⁺ counter ions, has a Mₙ as measured by NMR spectroscopy within a range of from 2000 to 2500 Da, preferably within a range of 2100 and 2300 Da.

Heparin according to the embodiments can be provided as a pharmaceutically acceptable salt of heparin. Pharmaceutically acceptable salt of heparin refers to a salt of dextran sulfate having the effects as disclosed herein and not being deleterious to the recipient thereof at the administered dose(s). Such pharmaceutically acceptable salts include e.g., a sodium or potassium salt of heparin. In a particular embodiment, the pharmaceutically acceptable salt is a sodium salt of heparin (heparin sodium salt).

In an embodiment, an effective amount of dextran sulfate, or the pharmaceutically acceptable salt thereof, and an effective amount of heparin, or the pharmaceutically acceptable salt thereof, are administered to the subject. Effective amount as used herein relates to a therapeutically effective amount of dextran sulfate, or the pharmaceutically acceptable salt thereof, and of heparin, or the pharmaceutically acceptable salt thereof, capable of causing a medical effect related to treatment of thromboinflammation when administered to the subject. Such a therapeutically effective amount is preferably an amount of dextran sulfate, or the pharmaceutically acceptable salt thereof, and of heparin, or the pharmaceutically acceptable salt thereof, that together are capable of inducing a change in at least one biomarker associated with thromboinflammation, such as a hematological parameter, a coagulation parameter, a complement parameter and/or a platelet parameter. The therapeutically effective amount of dextran sulfate, or the pharmaceutically salt thereof, and of heparin, or the pharmaceutically acceptable salt thereof, can be determined by the physician and may, optionally, be selected based on at least one among the sex of the subject, the weight of the subject, the age of the subject, the type of cell transplant, the type of organ or tissue transplant and the type of severity of the thromboinflammatory reaction.

Suitable dose ranges for the dextran sulfate, or the pharmaceutically acceptable salt thereof, of the embodiments may vary according to the size and weight of the subject, the condition for which the subject is treated, and other considerations. In particular for human subjects, a possible dosage range could be from 1 μg/kg to 150 mg/kg of body weight, preferably from 10 μg/kg to 100 mg/kg of body weight.

In preferred embodiments, dextran sulfate, or the pharmaceutically acceptable salt thereof, is formulated to be administered at a dosage in a range from 0.05 to 50 mg/kg of body weight of the subject, preferably from 0.05 or 0.1 to 40 mg/kg of body weight of the subject, and more preferably from 0.05 or 0.1 to 30 mg/kg, or 0.1 to 25 mg/kg or from 0.1 to 15 mg/kg or 0.1 to 10 mg/kg body weight of the subject. A currently preferred dosage of dextran sulfate, or the pharmaceutically acceptable salt thereof, is from 0.5 to 5 mg/kg body weight of the subject.

Suitable dose ranges for the heparin, or the pharmaceutically acceptable salt thereof, of the embodiments may vary according to the size and weight of the subject, the condition for which the subject is treated, and other considerations. In particular for human subjects, a possible dosage range could be from 25 IE/kg to 750 IE/kg body weight of the subject and day, preferably from 50 IE/kg to 600 IE/kg body weight of the subject and day, and more preferably from 100 IE/kg to 500 IE/kg body weight of the subject and day.

Administration of the combination of dextran sulfate, or the pharmaceutically acceptable salt thereof, and heparin, or the pharmaceutically acceptable salt thereof, does not necessarily have to be limited to treatment of thromboinflammation but could alternatively, or in addition, be used for prophylaxis. In other words, the combination of the embodiments could be administered to a subject having an increased risk of developing thromboinflammation.

Treatment of thromboinflammation also encompasses inhibition of thromboinflammation. Inhibition of thromboinflammation as used herein implies that the combination of dextran sulfate, or the pharmaceutically acceptable salt thereof, and heparin, or the pharmaceutically acceptable salt thereof, reduces the symptoms and effects of the condition even though a 100% treatment or cure does not necessarily occur. For instance, inhibition of thromboinflammation may involve a reduction in complement activation, a normalization of WBC counts, a reduction in clotting, a reduction in platelet aggregation, a reduction in formation of C5b-9 complex on platelets and/or a reduction in platelet binding to cell or organ transplant.

In an embodiment, the combination is formulated for simultaneous administration of dextran sulfate, or the pharmaceutically acceptable salt thereof, and heparin, or the pharmaceutically acceptable salt thereof. Hence, in this embodiment, the dextran sulfate, or the pharmaceutically acceptable salt thereof, and heparin, or the pharmaceutically acceptable salt thereof, are simultaneously administered to a subject. In such an embodiment, the dextran sulfate, or the pharmaceutically acceptable salt thereof, and heparin, or the pharmaceutically acceptable salt thereof, could be administered as a mixture to the subject. Alternatively, the dextran sulfate, or the pharmaceutically acceptable salt thereof, and heparin, or the pharmaceutically acceptable salt thereof, are administered separately to the patient either at different sites or at the same site but the administrations are given substantially simultaneously or at least overlapping in time. For instance, if both dextran sulfate, or the pharmaceutically acceptable salt thereof, and heparin, or the pharmaceutically acceptable salt thereof, are administered through infusion to a subject, the two infusions may be scheduled to start and end simultaneously, to start simultaneously but end at different points in time, to end simultaneously but start at different points in time, or indeed start and end at different points in time but being infused at least partly in parallel.

In another embodiment, the combination is formulated for sequential administration of dextran sulfate, or the pharmaceutically acceptable salt thereof, and the heparin, or pharmaceutically acceptable salt thereof. Sequential administration as used herein means that the dextran sulfate, or the pharmaceutically acceptable salt thereof, and heparin, or the pharmaceutically acceptable salt thereof, are administered at different points in time. For instance, dextran sulfate, or the pharmaceutically acceptable salt thereof, may be administered prior to administration of heparin, or the pharmaceutically acceptable salt thereof, or heparin, or the pharmaceutically acceptable salt thereof, may be administered prior to administration of dextran sulfate, or the pharmaceutically acceptable salt thereof. If any of dextran sulfate, or the pharmaceutically acceptable salt thereof, and heparin, or the pharmaceutically acceptable salt thereof, is scheduled for administration at multiple time instances, then these multiple administrations could be preceded, followed by and/or interrupted by one or more administrations of the other of dextran sulfate, or the pharmaceutically acceptable salt thereof, and heparin, or the pharmaceutically acceptable salt thereof.

It is also possible to combine at least one simultaneous administration of dextran sulfate, or the pharmaceutically acceptable salt thereof, and heparin, or the pharmaceutically acceptable salt thereof with at least one sequential administration of dextran sulfate, or the pharmaceutically acceptable salt thereof, and heparin, or the pharmaceutically acceptable salt thereof.

In an embodiment, a mixture of dextran sulfate, or the pharmaceutically acceptable salt thereof, and heparin, or the pharmaceutically acceptable salt thereof, dextran sulfate, or the pharmaceutically acceptable salt thereof, and/or heparin, or the pharmaceutically acceptable salt thereof, is formulated for systemic administration to the subject. In an embodiment, the mixture, dextran sulfate, or the pharmaceutically acceptable salt thereof, and/or heparin, or the pharmaceutically acceptable salt thereof, is formulated for parenteral administration as an example of systemic administration.

Examples of parenteral administration routes include intravenous (i.v.) administration, intra-arterial administration, intra-muscular administration, intracerebral administration, intracerebroventricular administration, intrathecal administration and subcutaneous (s.c.) administration.

In an embodiment, the mixture, dextran sulfate, or the pharmaceutically acceptable salt thereof, and/or heparin, or the pharmaceutically acceptable salt thereof, is preferably formulated for intravenous (i.v.) or subcutaneous (s.c.) administration to the subject. Accordingly, i.v. and s.c. administration are preferred examples of systemic administration. In a particular embodiment, dextran sulfate, or the pharmaceutically acceptable salt thereof, is formulated for s.c. administration to the subject and heparin, or the pharmaceutically acceptable salt thereof, is formulated for i.v. or s.c. administration.

In an embodiment, the mixture, dextran sulfate, or the pharmaceutically acceptable salt thereof, and/or heparin, or the pharmaceutically acceptable salt thereof, is formulated as an injection solution, preferably an i.v. or s.c. injection solution. Thus, the mixture, dextran sulfate, or the pharmaceutically acceptable salt thereof, and/or heparin, or the pharmaceutically acceptable salt thereof, is preferably formulated as an injection solution with a selected solvent or excipient.

In an embodiment, dextran sulfate, or the pharmaceutically acceptable salt thereof is formulated as an aqueous injection solution, preferably as an aqueous i.v. or s.c. injection solution. The solvent is advantageously an aqueous solvent, such as a buffer solution. A non-limiting example of such a buffer solution is a citric acid buffer, such as citric acid monohydrate (CAM) buffer, or a phosphate buffer, such as phosphate buffered saline (PBS). Also non-buffered solutions are possible, including aqueous injection solutions, such as saline, i.e., NaCl (aq). Furthermore, other buffer systems than CAM and phosphate buffers could be used if a buffered solution is desired. For instance, dextran sulfate, or the pharmaceutically acceptable salt thereof, can be dissolved in saline, such as 0.9% NaCl saline, and then optionally buffered with 75 mM CAM and adjusting the pH to about 5.9 using sodium hydroxide.

In an embodiment, heparin, or the pharmaceutically acceptable salt thereof, is formulated as an injection solution, preferably an i.v. or s.c. injection solution using, in an illustrative example, benzyl alcohol as a solvent. The injection solution may optionally include excipients, such as anti-fungal agents, for instance methylparaben (E218) and/or propylparaben (E216), buffer agent, such as sodium citrate, and/or pH adjusting agent, such as hydrochloric acid. Heparin, or the pharmaceutically acceptable salt thereof, dissolved in benzyl alcohol may, in turn, be dissolved in another solvent prior to administration, such as saline.

The embodiments are not limited to injections and other administration routes can alternatively be used including nasal, buccal, dermal, tracheal, bronchial, or topical administration. Also, local administration of the mixture, dextran sulfate, or the pharmaceutically acceptable salt thereof, and/or heparin, or the pharmaceutically acceptable salt thereof, is possible, such as at the site of transplantation of the cell or organ transplant.

The active compound(s), dextran sulfate, or the pharmaceutically acceptable salt thereof, and/or heparin, or the pharmaceutically acceptable salt thereof, is(are) then formulated with a suitable excipient, solvent or carrier that is selected based on the particular administration route.

Carrier refers to a substance that serves as a vehicle for improving the efficiency of delivery and/or the effectiveness of the mixture, dextran sulfate, or the pharmaceutically acceptable salt thereof and/or heparin, or the pharmaceutically acceptable salt thereof.

Excipient refers to a pharmacologically inactive substance that is formulated in combination with the mixture, dextran sulfate, or the pharmaceutically acceptable salt thereof, and/or heparin, or the pharmaceutically acceptable salt thereof, and includes, for instance, bulking agents, fillers, diluents and products used for facilitating drug absorption or solubility or for other pharmacokinetic considerations.

It is also possible to add the mixture, dextran sulfate, or the pharmaceutically acceptable salt thereof, and/or heparin, or the pharmaceutically acceptable salt thereof, to the cell or organ transplant to thereby administer the cell transplant together with the mixture, dextran sulfate, or the pharmaceutically acceptable salt thereof, and/or heparin, or the pharmaceutically acceptable salt thereof, or to transplant the organ together with the mixture, dextran sulfate, or the pharmaceutically acceptable salt thereof, and/or heparin, or the pharmaceutically acceptable salt thereof.

Such an administration of the cell transplant or transplantation of the organ transplant together with the mixture, dextran sulfate, or the pharmaceutically acceptable salt thereof, and/or heparin, or the pharmaceutically acceptable salt thereof may be combined with any of the previously described administrations of the mixture, dextran sulfate, or the pharmaceutically acceptable salt thereof, and/or heparin, or the pharmaceutically acceptable salt thereof.

The mixture, dextran sulfate, or the pharmaceutically acceptable salt thereof, and/or heparin, or the pharmaceutically acceptable salt thereof, can be administered at a single administration occasion, such as in the form of a single injection or bolus injection. This bolus dose can be injected quite quickly to the subject but is advantageously infused over time so that the mixture, dextran sulfate, or the pharmaceutically acceptable salt thereof, and/or heparin, or the pharmaceutically acceptable salt thereof, is infused over a few minutes of time to the subject, such as during 5 to 10 minutes or more. Also slow-release formulations of the mixture, dextran sulfate, or the pharmaceutically acceptable salt thereof, and/or heparin, or the pharmaceutically acceptable salt thereof, can be used in order to achieve a prolonged release thereof.

Alternatively, the mixture, dextran sulfate, or the pharmaceutically acceptable salt thereof, and/or heparin, or the pharmaceutically acceptable salt thereof, can be administered at multiple, i.e., at least two, occasions during a treatment period. Thus, the mixture, dextran sulfate, or the pharmaceutically acceptable salt thereof, and/or heparin, or the pharmaceutically acceptable salt thereof, could be administered once or at multiple times per day, once or at multiple times per week, once or at multiple times per month as illustrative examples.

In a particular embodiment, dextran sulfate, or the pharmaceutically acceptable salt thereof, is formulated for administration at 1-14 times, preferably 1-7 times, a week for one or multiple consecutive weeks, such as at least 2-5 consecutive weeks. In a particular embodiment, dextran sulfate, or the pharmaceutically acceptable salt thereof, is formulated for administration once or twice a day for multiple days, such as multiple consecutive days, e.g., 2-14 days.

In a particular embodiment, heparin, or the pharmaceutically acceptable salt thereof, is formulated for administration at 1-14 times, preferably 1-7 times, a week for one or multiple consecutive weeks. In a particular embodiment, heparin, or the pharmaceutically acceptable salt thereof, is formulated for administration once to six times a day for multiple days, such as multiple consecutive days, e.g., 2-14 days.

It is also possible combine a bolus injection of the mixture, dextran sulfate, or the pharmaceutically acceptable salt thereof, and/or heparin, or the pharmaceutically acceptable salt thereof, with one or more additional administrations of the mixture, dextran sulfate, or the pharmaceutically acceptable salt thereof and/or heparin, or the pharmaceutically acceptable salt thereof.

In an embodiment, the subject is a mammalian subject, preferably a primate, and more preferably a human subject. Although the embodiments are in particular directed towards treatment of thromboinflammation in human subjects, the embodiments may also, or alternatively, be used in veterinary applications. Non-limiting example of animal subjects include non-human primate, cat, dog, pig, horse, mouse, rat, goat, guinea pig, sheep and cow.

In an embodiment, the thromboinflammation is selected from the group consisting of IBMIR and IRI. In a particular embodiment, the thromboinflamamtion is IBMIR and the combination comprising an effective amount of dextran sulfate, or a pharmaceutically acceptable salt thereof, and an effective amount of heparin, or a pharmaceutically acceptable salt thereof, is for use in treatment of IBMIR. In another particular embodiment, the thromboinflamamtion is IRI and the combination comprising an effective amount of dextran sulfate, or a pharmaceutically acceptable salt thereof, and an effective amount of heparin, or a pharmaceutically acceptable salt thereof, is for use in treatment of IRI.

The invention also relates to the use of a combination comprising an effective amount of dextran sulfate, or a pharmaceutically acceptable salt thereof, and an effective amount of heparin, or a pharmaceutically acceptable salt thereof, in the manufacture of a medicament for treatment or prevention of thromboinflammation.

The invention also defines a method for treating or preventing thromboinflammation. The method comprises administering a combination comprising dextran sulfate, or a pharmaceutically acceptable salt thereof, and heparin, or a pharmaceutically acceptable salt thereof, to a subject suffering from thromboinflammation.

In an embodiment, the method comprises simultaneously administering dextran sulfate, or the pharmaceutically acceptable salt thereof, and heparin, or the pharmaceutically acceptable salt thereof to the subject.

In another embodiment, the method comprises sequentially administering dextran sulfate, or the pharmaceutically acceptable salt thereof, and heparin, or the pharmaceutically acceptable salt thereof to the subject.

The present invention also relates to a transplant composition comprising a transplant, dextran sulfate, or the pharmaceutically acceptable salt thereof, and heparin, or the pharmaceutically acceptable salt thereof.

In an embodiment, the transplant composition is a cell transplant composition comprising a cell transplant, dextran sulfate, or the pharmaceutically acceptable salt thereof, and heparin, or the pharmaceutically acceptable salt thereof.

The cell transplant composition may also comprise a solvent or solution comprising dextran sulfate, or the pharmaceutically acceptable salt thereof, and heparin, or the pharmaceutically acceptable salt thereof, and comprising the cell transplant.

In another embodiment, the transplant composition is an organ transplant composition comprising an organ or tissue transplant, dextran sulfate, or the pharmaceutically acceptable salt thereof, and heparin, or the pharmaceutically acceptable salt thereof.

The organ transplant composition preferably also comprises an organ preservation solution comprising dextran sulfate, or the pharmaceutically acceptable salt thereof, and heparin, or the pharmaceutically acceptable salt thereof. The organ or tissue transplant is then immersed in the organ preservation solution.

Illustrative, but non-limiting, examples of such organ preservation solutions include a histidine-tryptophan-ketoglutarate (HTK) solution, a citrate solution, a University of Wisconsin (UW) solution, a Collins solution, a Celsior solution, a Kyoto University solution and an Institut Georges Lopez-1 (IGL-1) solution.

Illustrative, but non-limiting, examples of organ transplants include kidney, heart, pancreas, liver, lung, uterus, urinary bladder, thymus, intestine and spleen.

A blood loop model is an effective in vitro model for studying the IBMIR reaction evoked by a cell transplant in direct contact with blood and the beneficial effects seen with a combination of dextran sulfate, or a pharmaceutically acceptable salt thereof, and heparin, or a pharmaceutically acceptable salt thereof, according to the invention.

In a typical embodiment, the blood loop model uses tubing, such as polyvinyl chloride (PVC) tubing, that are closed using a connector to form a tubing loop. The tubing and preferably also the connector are heparinized, i.e., furnished with immobilized heparin. For instance, heparinized PVC tubing, with an inner diameter of from 2 mm up to 10 mm, such as from 3 mm up to 8 mm, preferably from 3.5 mm up to 7.5 mm, and a length of from 100 mm up to 600 mm, such as from 200 up to 500 mm, preferably from 300 up to 400 mm, could be used.

In an embodiment, heparin from Corline (CHC™, Corline, Uppsala, Sweden) is used to provide all material in contact with blood with at least one layer, such as two layers, of immobilized heparin in order to minimize material induced platelet activation. The CHC™ consists of heparin conjugates (M_{w}˜13 kDa), in which heparin is covalently bound to a polyamine carrier, approximately 70 moles of heparin per mole carrier of protein (Biomaterials 24(23): 4153-4159 (2003)).

The closed blood loop is preferably held at 37° C. and kept in motion at a predetermined speed, such as using a rocking device. The predetermined speed, such as vertical rotation of the rocking device, is preferably selected to mimic blood flow in a patient body. For instance, a rocking device that is vertically rotated at 30 rpm generates a blood flow of 45 ml/min, which mimics the portal venous flow.

The volume of blood, preferably ABO-matched whole blood, added to the blood loop depends on the internal volume of the tubing loop. In illustrative embodiments, the volume of blood is selected within an interval of from 0.5 ml up to 15 ml, preferably from 1 ml up to 10 ml, such as from 1.5 ml up to 8 ml.

The whole blood added to the blood loop is preferably whole mammalian blood, and more preferably whole human blood. However, if the blood loop model is to be used be used in veterinary applications, the whole mammalian blood could, for instance, be selected from the group consisting of blood from non-human primates, cats, dogs, pigs, horses, mice, rats, goats, guinea pigs, sheep or cows.

An aspect of the invention relates to an in vitro IBMIR model method. The method comprises adding in vitro test cells to a blood loop comprising whole blood to trigger IBMIR. The method also comprises adding in vitro dextran sulfate, or a pharmaceutically acceptable salt thereof, to the blood loop. The method further comprises adding in vitro heparin, or a pharmaceutically acceptable salt thereof, to the whole blood in the blood loop. The method additionally comprises determining an effectiveness of a combination of dextran sulfate, or the pharmaceutically acceptable salt thereof, and heparin, or the pharmaceutically acceptable salt thereof, to inhibit IBMIR by determining at least one hematological parameter, at least one coagulation parameter, at least one complement parameter and at least one platelet parameter.

The addition of the test cells, dextran sulfate, or the pharmaceutically acceptable salt thereof, and heparin, or the pharmaceutically acceptable salt thereof, could be performed sequentially in any order or at least partly in parallel.

According to the invention, adding in vitro heparin, or the pharmaceutically acceptable salt thereof, comprises adding heparin, or the pharmaceutically acceptable salt thereof, to the whole blood in the blood loop. Hence, the heparin, or the pharmaceutically acceptable salt thereof, added in the method is not any heparin used to heparinize the tubing and/or connector used in the tubing loop. Hence, heparin, or the pharmaceutically acceptable salt thereof, added according to the method is not bond to any surface to the blood loop, i.e., is not bond to the inner surface of the tubing and/or connector. In an embodiment, heparin, or the pharmaceutically acceptable salt thereof, is soluble heparin, or the pharmaceutically acceptable salt thereof. Hence, the heparin, or the pharmaceutically acceptable salt thereof, is present in the whole blood in the blood loop, such as dispersed or dissolved in the whole blood.

In an embodiment, the method also comprises adding in vitro test cells to a control blood loop comprising whole blood and determining at least one hematological parameter, at least one coagulation parameter, at least one complement parameter and at least one platelet parameter in the control blood loop. Hence, a blood loop lacking any added dextran sulfate, or the pharmaceutically acceptable salt thereof, and heparin, or the pharmaceutically acceptable salt thereof, but comprising the in vitro added test cells is used as control. In such a case, the at least one hematological parameter, at least one coagulation parameter, at least one complement parameter and at least one platelet parameter as determined in the blood loop comprising in vitro added dextran sulfate, or the pharmaceutically acceptable salt thereof, and heparin, or the pharmaceutically acceptable salt thereof, are compared to corresponding parameters as determined in the control blood loop. The effectiveness of the combination of dextran sulfate, or the pharmaceutically acceptable salt thereof, and heparin, or the pharmaceutically acceptable salt thereof, to inhibit IBMIR can then be determined based on the comparison.

In an embodiment, the hematological parameter is selected from the group consisting of white blood cell (WBC) count, neutrophil count, monocyte count, eosinophil count and a combination thereof.

In an embodiment, the coagulation parameter is selected from the group consisting of activated partial thromboplastin time (APTT), blood clot size, hemolysis and a combination thereof.

In an embodiment, the complement parameter is selected from the group consisting of C3a, C5a and combination thereof.

In an embodiment, the platelet parameter is selected from the group consisting of proportion of platelets bound to the test cells, proportion of platelets bound to monocytes, proportion of monocytes bond to granulocytes, proportion of C5b-9 positive platelets and a combination thereof.

In an embodiment, dextran sulfate, or the pharmaceutically acceptable salt thereof, is added at a final concentration of from 0.025 up to 2 mg/ml, preferably of from 0.05 up to 1 mg/ml, and more preferably of from 0.1 up to 0.5 mg/ml, such as about 0.2 mg/ml.

In an embodiment, heparin, or the pharmaceutically acceptable salt thereof, is added at a final concentration of from 0.025 up to 2 IE/ml, preferably of from 0.05 up to 1 IE/ml, and more preferably of from 0.2 up to 0.75 IE/ml, such as from about 0.2 up to 0.5 IE/ml.

Another aspect of the invention relates to an in vitro IBMIR model kit. The kit comprises a tubing and a connector configured to interconnect the tubing to form a tubing loop. The kit also comprises dextran sulfate, or a pharmaceutically acceptable salt thereof, and heparin, or a pharmaceutically acceptable salt thereof. The heparin, or the pharmaceutically salt thereof, is not immobilized to an inner surface of the tubing or the connector.

In an embodiment, the kit also comprises test cells.

In an embodiment, the tubing is a heparinized tubing. In an embodiment, the connector is a heparinized connector.

In an embodiment, the kit also comprises instructions for adding test cells, dextran sulfate, or a pharmaceutically acceptable salt thereof, and heparin, or a pharmaceutically acceptable salt thereof, to whole blood contained in the tubing. In a particular embodiment, the kit further comprises instructions for determining an effectiveness of a combination of dextran sulfate, or the pharmaceutically acceptable salt thereof, and heparin, or the pharmaceutically acceptable salt thereof, to inhibit IBMIR by determining at least one hematological parameter, at least one coagulation parameter, at least one complement parameter and at least one platelet parameter.

### Examples

The present Example studied the effects of dextran sulfate, and combinations of dextran sulfate and heparin, and WI-38 cells on a number of hematological and immunological parameters in an IBMIR blood loop system.

Materials and Methods Dextran sulfate (IBsolvMIR®, Tikomed AB, Viken, Sweden, WO 2016/076780), heparin (Heparin LEO®, LEO Pharma, cat. No. 585679) and WI-38 cells (American Type Culture Collection (ATCC)) were administered to blood loops as shown in Table 1. Vehicle was an aqueous 0.9% NaCl solution. Each loop contained in total 2 ml of freshly taken human whole blood from three blood donors. Reagents were added to the loops according to Table 1 and the loops were set to rotate at 37° C. Blood samples were taken at the zero, 15 min, 1 hour and 4 hour time points.

TABLE 1
experimental setup in the blood loop system

| Loop | Reagent | Final conc. | Reagent | Final conc. | |
|---|---|---|---|---|---|
| # | #1 | in blood | #2 | in blood | Heparin |
| 1 | Vehicle | | Vehicle | | 0 |
| 2 | Vehicle | | IBsolvMIR ® | 0.2 mg/ml | 0 |
| 3 | WI-38 | 1 × 10⁵/2 ml | Vehicle | | 0 |
| 4 | WI-38 | 1 × 10⁵/2 ml | IBsolvMIR ® | 0.2 mg/ml | 0 |
| 5 | Vehicle | | Vehicle | | 0.2 U/ml |
| 6 | Vehicle | | IBsolvMIR ® | 0.2 mg/ml | 0.2 U/ml |
| 7 | WI-38 | 1 × 10⁵/2 ml | Vehicle | | 0.2 U/ml |
| 8 | WI-38 | 1 × 10⁵/2 ml | IBsolvMIR ® | 0.2 mg/ml | 0.2 U/ml |
| 9 | Vehicle | | Vehicle | | 0.5 U/ml |
| 10 | Vehicle | | IBsolvMIR ® | 0.2 mg/ml | 0.5 U/ml |
| 11 | WI-38 | 1 × 10⁵/2 ml | Vehicle | | 0.5 U/ml |
| 12 | WI-38 | 1 × 10⁵/2 ml | IBsolvMIR ® | 0.2 mg/ml | 0.5 U/ml |

Hematology Analysis

Blood samples were extracted from loops at the zero and 1 hour time points and platelets, red blood cells and white blood cells were automatically counted using a Sysmex XN-L 350 Hematology Analyzer.

Coagulation and Hemolysis

Blood clot size and hemolysis were observed for the three donors (donor 1 (D1), donor 2 (D2) and donor 3 (D3)) at three blood sampling times 15 min, 1 hour and 4 hours.

Cytokine Analysis

Blood was extracted from loops at the zero and 4 hour time points and cytokines were analyzed by electrochemiluminescence (ECL) in multiplex mode using MESO QuickPlex SQ 120 (Mesoscale Discovery).

Flow Cytometry Analysis

Tables 2 and 3 list the cell staining agents used in the flow cytometry analysis.

TABLE 2
staining of non-lysed blood (platelets)

| Cell staining agent | Source |
|---|---|
| CD41 - BV510 | Biolegend, cat. no. 303736 |
| CD62P - PE | Biolegend, cat. no. 304906 |
| CD14 - PE/Cy7 | Biolegend, cat. no. 301814 |
| CD66b - PerCP/Cy5.5 | Biolegend, cat. no. 305108 |
| CD45 - BV421 | Biolegend, cat. no. 304032 |
| Anti-C5b-9 (primary antibody; IgG2) | Abcam, cat. no. ab66768 |
| Anti-IgG2 - AF488 (secondary | Southern Biotech, cat. no. 9070-30 |
| antibody) | |
| Fibroblast marker - APC | Miltenyi, cat. no. 130-100-132 |

TABLE 3
staining of lysed blood

| Cell staining agent | Source |
|---|---|
| CD19 - BV650 | Biolegend, cat. no. 302238 |
| CD3 - BV510 | Biolegend, cat. no. 300448 |
| CD14 - PE/Cy7 | Biolegend, cat. no. 301814 |
| CD66b - PerCP/Cy5.5 | Biolegend, cat. no. 305108 |
| CD11b -APC | Biolegend, cat. no. 301410 |
| CD69 - BV421 | Biolegend, cat. no. 310930 |
| TF (CD142) - PE | Biolegend, cat. no. 365203 |
| CD19 - BV650 | Biolegend, cat. no. 302238 |
| CD3 - BV510 | Biolegend, cat. no. 300448 |
| CD14 - PE/Cy7 | Biolegend, cat. no. 301814 |
| CD66b - PerCP/Cy5.5 | Biolegend, cat. no. 305108 |
| Anti-C5b-9 (primary antibody; IgG2) | Abcam, cat. no. ab66768 |
| Anti-IgG2 - AF488 (secondary | Southern Biotech, cat. no. 9070-30 |
| antibody) | |
| Fibroblast marker - APC | Miltenyi, cat. no. 130-100-132 |
| Fixable Viability Dye eFluor 780 | Invitrogen, cat. no. 65-0865-18 |
| Annexin V eFluor 450 | Invitrogen, cat. no. 88-8006-74 |

Fresh blood was collected and immediately mixed with ethylenediaminetetraacetic acid (EDTA) (zero time point samples) or added to loops followed by sampling and mixing with EDTA at the 1 hour time point. Data was analyzed using flow cytometry.

Complement analysis Complement split product C3a and C5a concentrations were measured by ELISA according to the manufacturer's instructions (RayBio® Human C3a and C5a ELISA Kit for serum, plasma, and cell culture supernatants by RayBiotech) at the zero, 15 min and 1 hour time points.

Results

The present Example investigated the effect of WI-38 and cytokine levels and complement in combination with IBsolvMIR® in a blood loop system. Vehicle, WI-38 (1×10⁵ cells), IBsolvMIR® (0.2 mg/ml) and heparin (no heparin, at 0.2 U/ml and 0.5 U/ml) were added to blood loops and blood was sampled after 15 min, 1 and 4 hours in the blood loop system.

Hematology Analysis

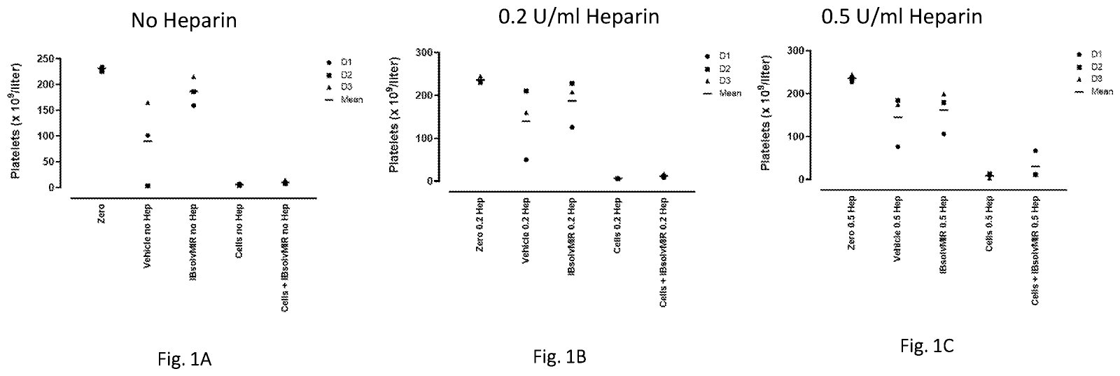

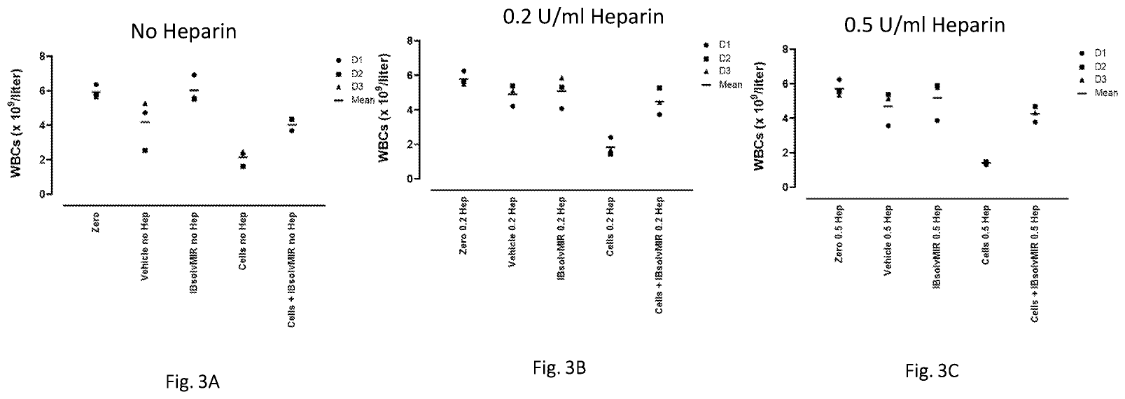

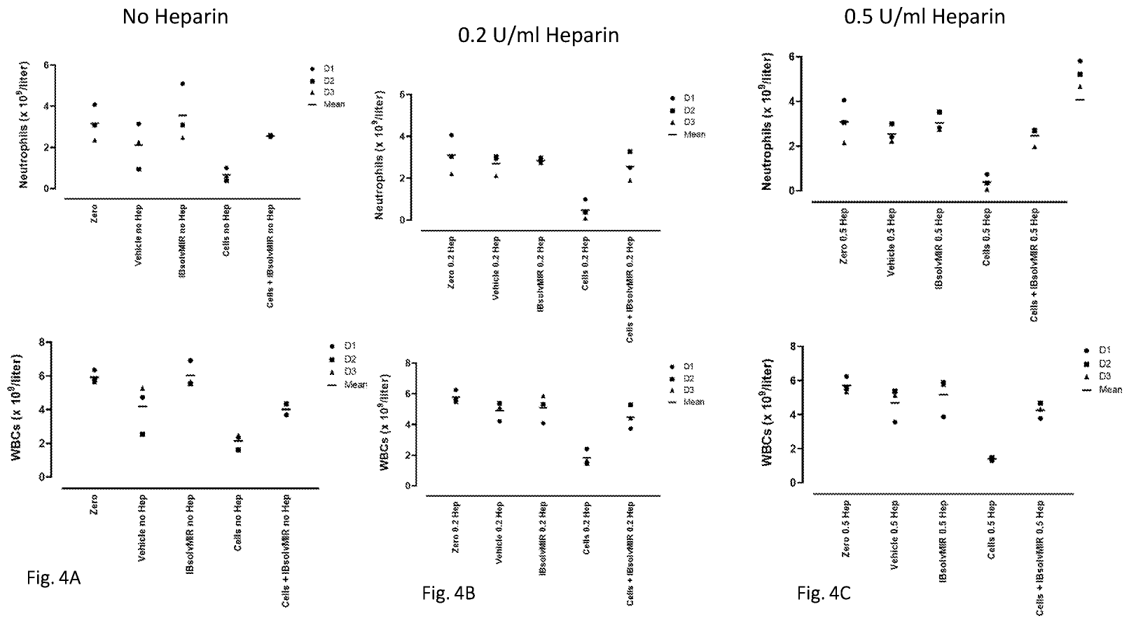

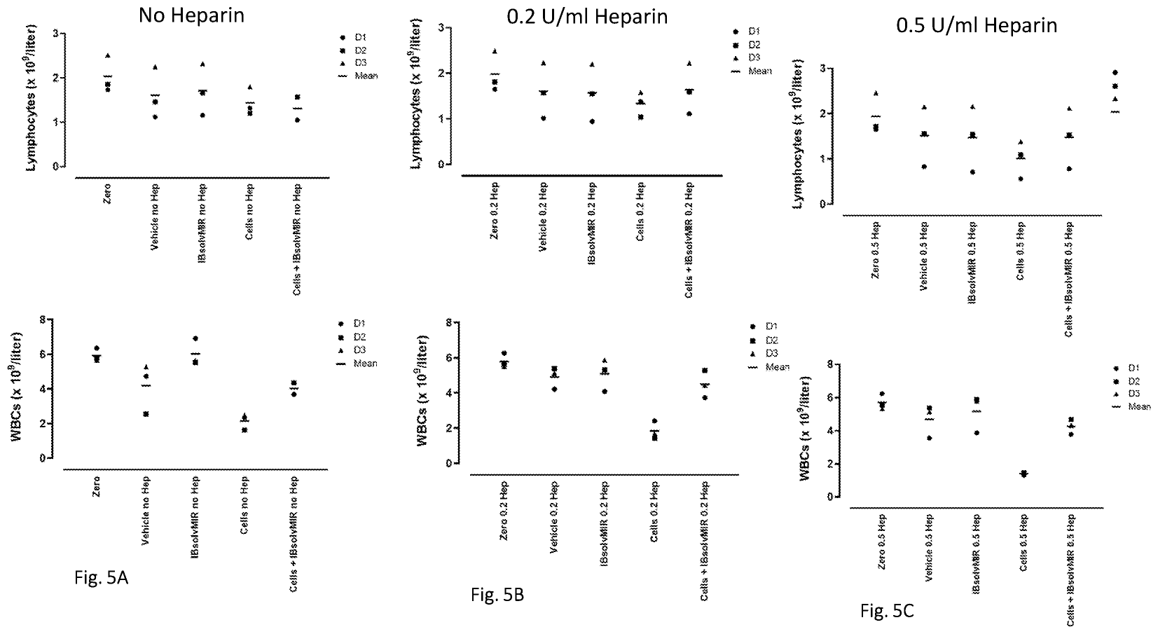

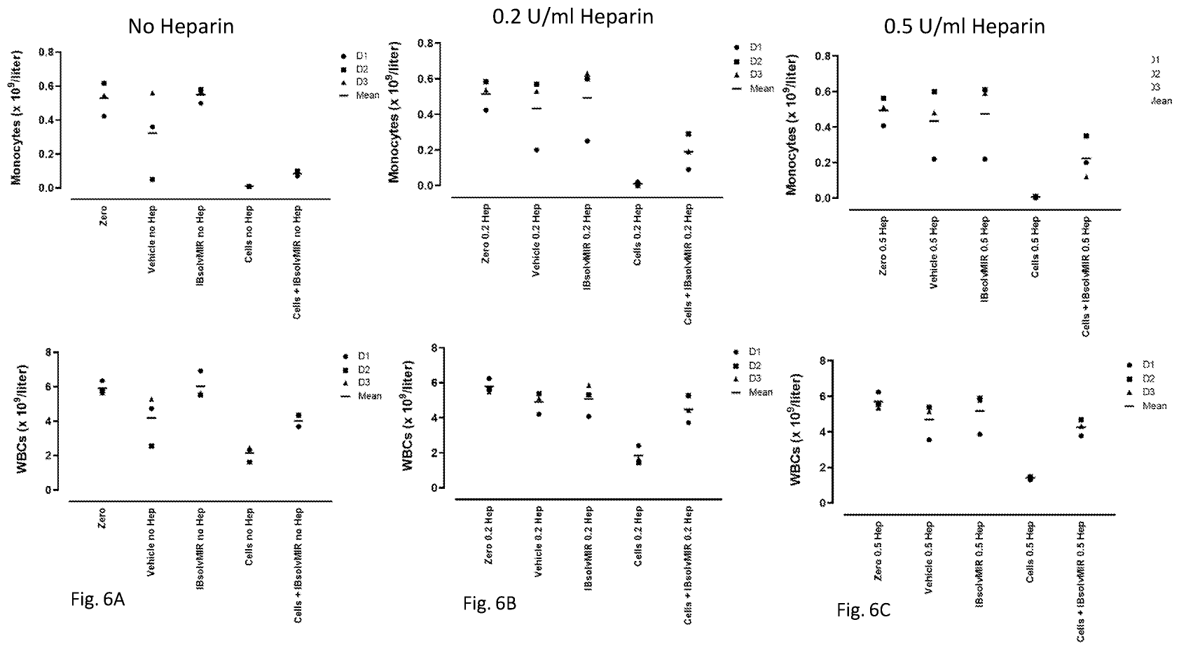

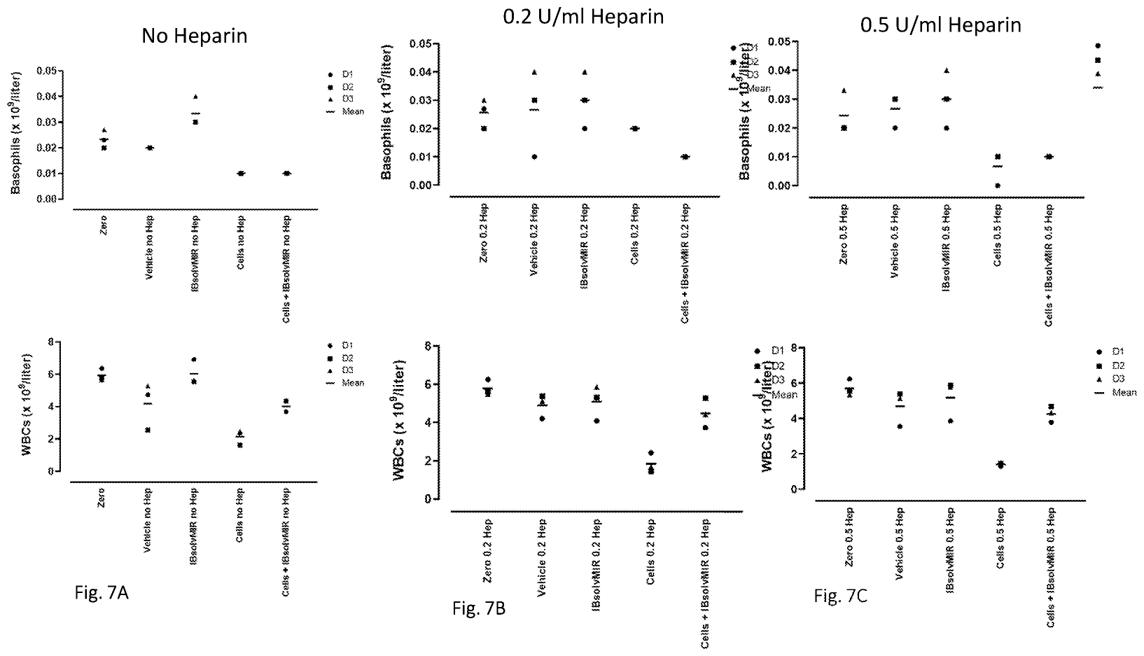

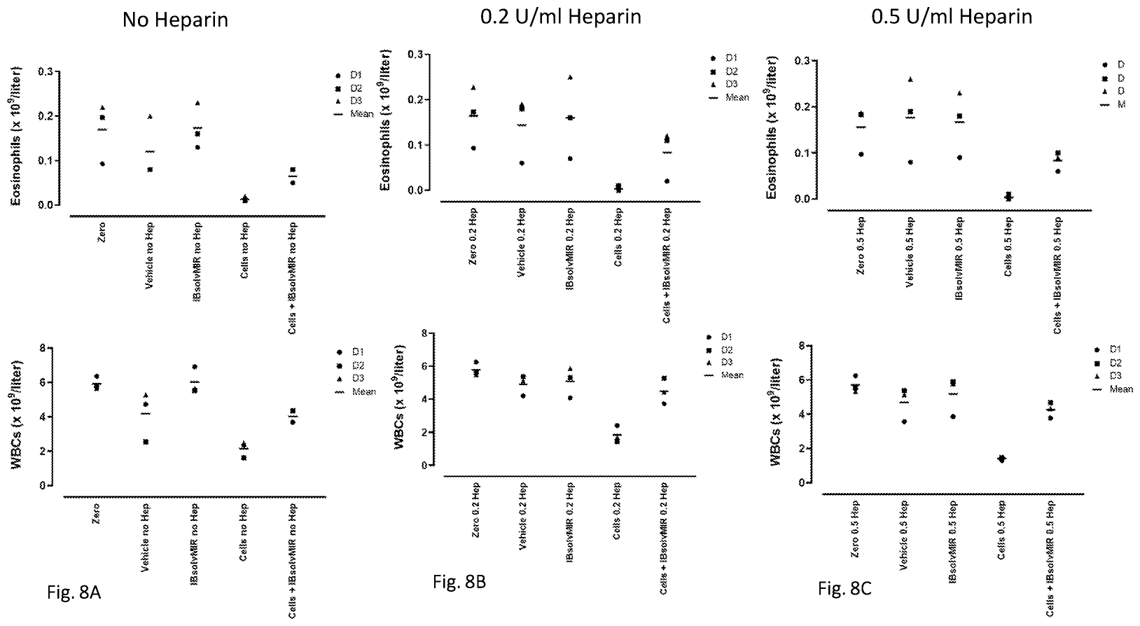

Platelet (PLT) counts decreased in vehicle but with large variability between donors and groups (FIGS. 1A to 1C). A clear decrease in PLT and WBC counts compared to vehicle was noted in all groups with WI-38 cells (FIGS. 3A to 3C). Co-administration of IBsolvMIR® with WI-38 cells or IBsolvMIR® and heparin with WI-38 cells completely rescued the WBC decrease (FIGS. 3A to 3C). As is shown in FIGS. 4A to 8C, addition of WI-38 cells to the blood loops mainly affected the WBC types neutrophils, monocytes, basophils and eosinophils, whereas lymphocyte counts were less affected.

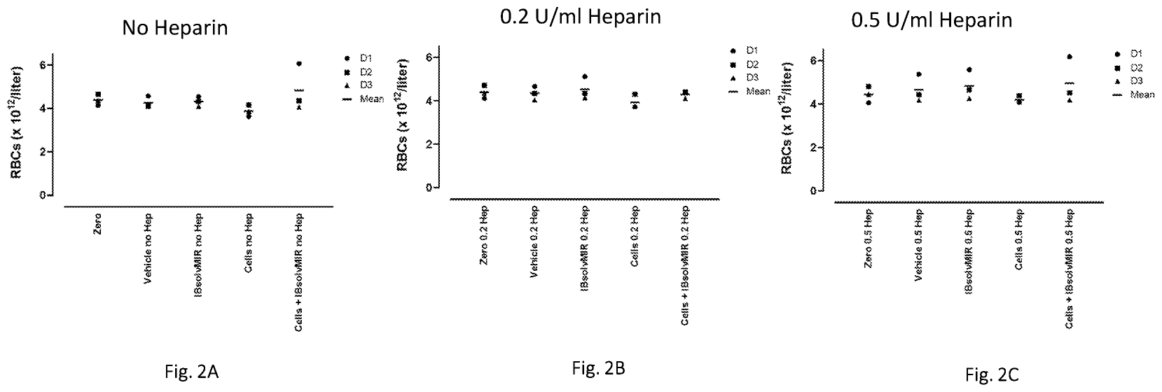

Red blood cell (RBC) counts were not affected by any of the reagents (FIGS. 2A to 2C).

Coagulation and Hemolysis

Clotting was observed with large clots (>5 mm) in virtually all WI-38-treated samples (loops 3, 7 and 11) in the absence of IBsolvMIR® as early as at 15 minutes whereas no clotting was observed in the WI-38+IBsolvMIR® at that time point, see Table 4. At one hour, medium-sized clots were observed in two WI-38+IBsolvMIR® loops. At 4 hours, clotting had been observed in all cell-treated loops and in 5/9 of the vehicle-treated loops. No clotting was observed in any of the IBsolvMIR® alone loops in any of the donors.

TABLE 4
clotting and hemolysis

| | Donor 1 | | | | Donor 2 | | | | Donor 3 | | | |
|---|---|---|---|---|---|---|---|---|---|---|---|---|
| Loop # | 15 min | 1 h | 4 h | Hem | 15 min | 1 h | 4 h | Hem | 15 min | 1 h | 4 h | Hem |
| 1 | | | 3 | | | 3* | 3 | | | | 2 | |
| 2 | | | | | | | | | | | | |
| 3 | 3* | 2-3 | 3 | | 3* | 2, 3* | 3 | | 3* | 3* | | |
| 4 | | | 3 | | | | 3 | + | | 2* | 3 | |
| 5 | | | 3 | | | | | | | | 1 | |
| 6 | | | | | | | | | | | | |
| 7 | 3* | 2-3 | 3 | | 2-3 | | 3 | | 3* | | | |
| 8 | | | 4 | | | | 3 | + | | 2* | 3 | |
| 9 | | | | | | | | | | | | |
| 10 | | | | | | | | | | | | |
| 11 | 3* | 2* | n.s. | + | 3* | 3* | | | 3* | | 3 | |
| 12 | | | 3 | | | 2* | 3 | + | | | 3 | |

Table 4 shows scoring of blood clot size and observed hemolysis for donors D1, D2 and D3 at the three blood sampling times 15 min, 1 and 4 hours. Clot scale: 1=Small clot observed (<2 mm), 2=Medium-sized clot observed (2-5 mm), 3=Large clot observed (>5 mm), * Clot removed from loop (not noted at 4 h when all blood was removed from loop), n.s.=no sample. Hemolysis: +=hemolysis observed in blood loop.

Cytokine Analysis

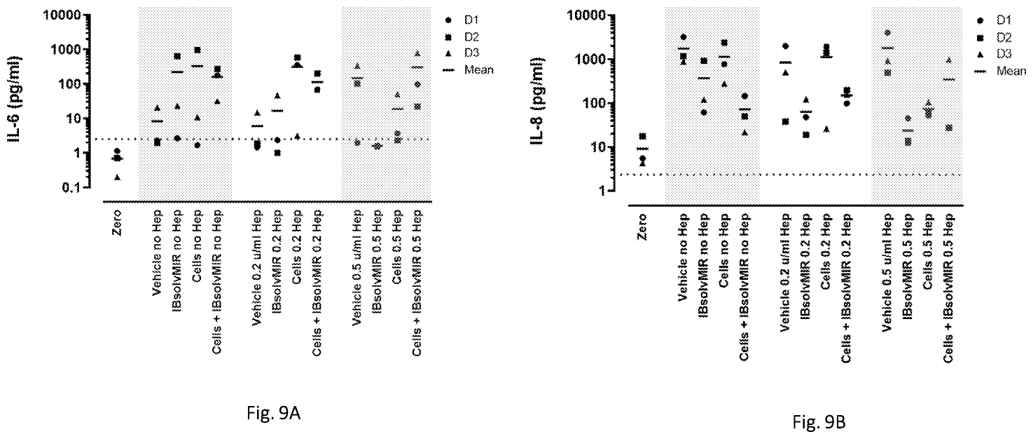

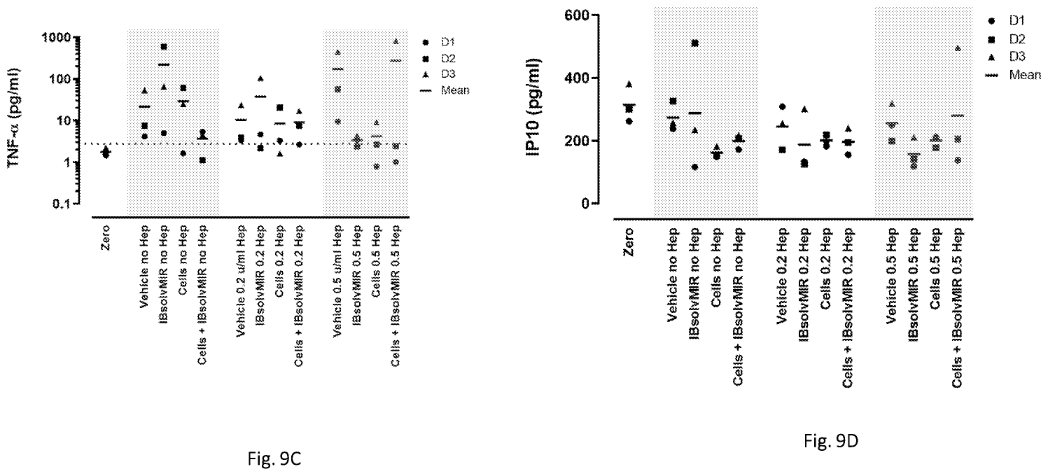

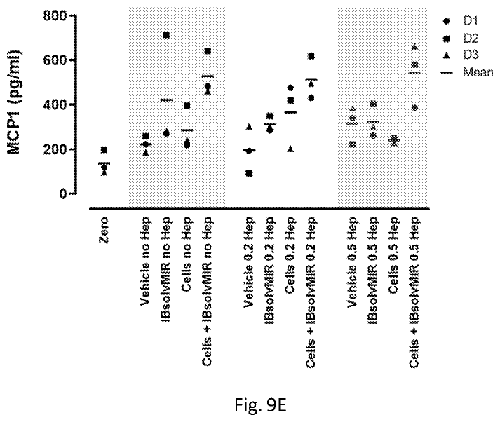

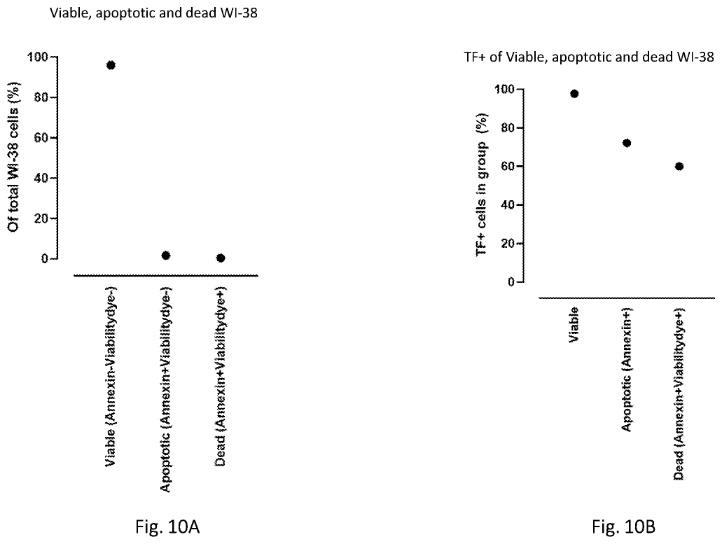

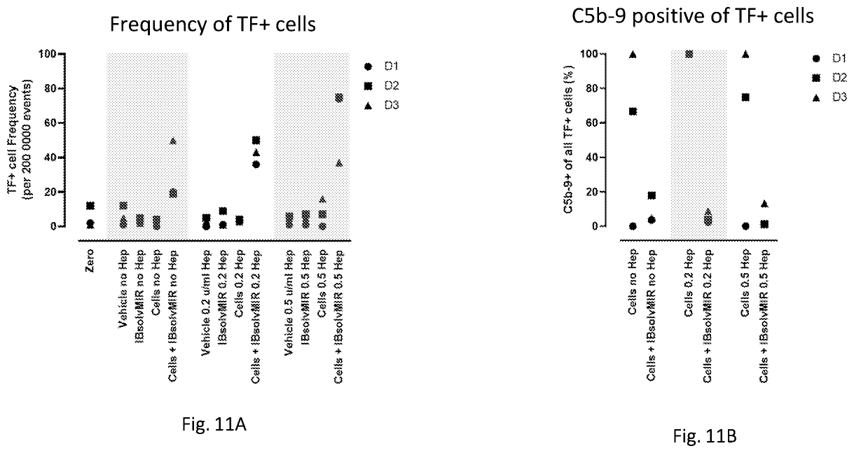

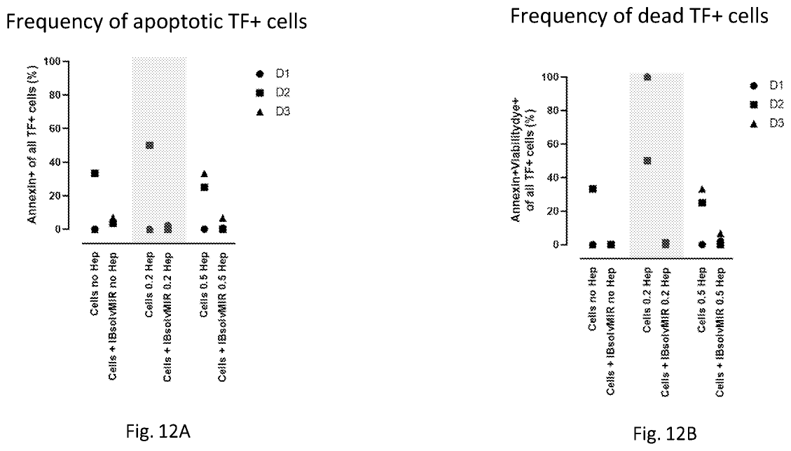

IL-6, IL-8, TNF-α, IP10 and MCP-1 cytokine release were measured at the zero and 4 hour time points. Background (vehicle) levels were low for IL-6 with no heparin and 0.2 U/ml heparin but donors D2 and D3 responded strongly (>100-fold) with IL-6 even with the vehicle at 0.5 U/ml heparin (FIG. 9A). The effects of IBsolvMIR® were ambiguous. A similar profile was seen for TNF-α with no heparin and 0.2 U/ml heparin and donors D2 and D3 causing a dramatic elevated vehicle response at 0.5 U/ml heparin (FIG. 9C). TNF-α in D3 was also very high in the WI-38 cells+IBsolvMIR® at 0.5 U/ml heparin group. These results agree well with the activation of both granulocytes and monocytes for that donor (FIG. 9C).

The vehicle responses for IL-8 were high in all three vehicle groups (FIG. 9B). In contrast to IL-6 and TNF-α, however, a clear IBsolvMIR® dependent reduction of vehicle and cell-induced IL-8 was observed (FIG. 9B).

The levels of IP10 and MCP-1 were similar between vehicle and zero time point samples (FIGS. 9D and 9E). IP10 levels were similar across the groups with high variations in IBsolvMIR® and no heparin (D2 was high) and WI-38 cells+IBsolvMIR® 0.5 U/ml heparin (D3 was high) (FIG. 9D). MCP-1 displayed a similar pattern but overall the IBsolvMIR® alone groups were higher than corresponding vehicle (FIG. 9E). WI-38 cells with IBsolvMIR® showed slightly increased levels of MCP-1 compared to the corresponding WI-38 alone group (FIG. 9E).

Flow Cytometry Analysis

WI-38 cells were found to be viable (96%) and mostly present as single cells (90.9%) (FIG. 10A). Almost the entire population of viable WI-38 were tissue factor (TF) positive (FIG. 10B). TF positive cell counts increased about ten-fold in all groups when comparing corresponding WI-38 treated groups with and without IBsolvMIR® (FIG. 11A). In all groups, the levels with WI-38 cells but without IBsolvMIR® were similar comparing zero and vehicle. The frequency of apoptotic (annexin +) and dead (annexin + and/or viability dye +) was lower in all IBsolvMIR® treated groups compared to corresponding WI-38 treated without IBsolvMIR® (FIGS. 12A and 12B). Also, complement activation (C5b-9) on TF positive cells was lower in all groups with IBsolvMIR® compared to WI-38 alone (FIG. 11B).

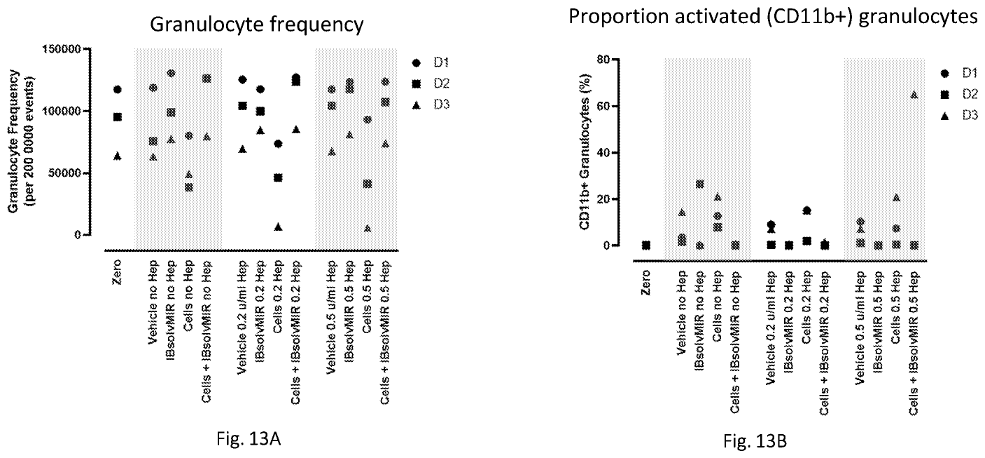

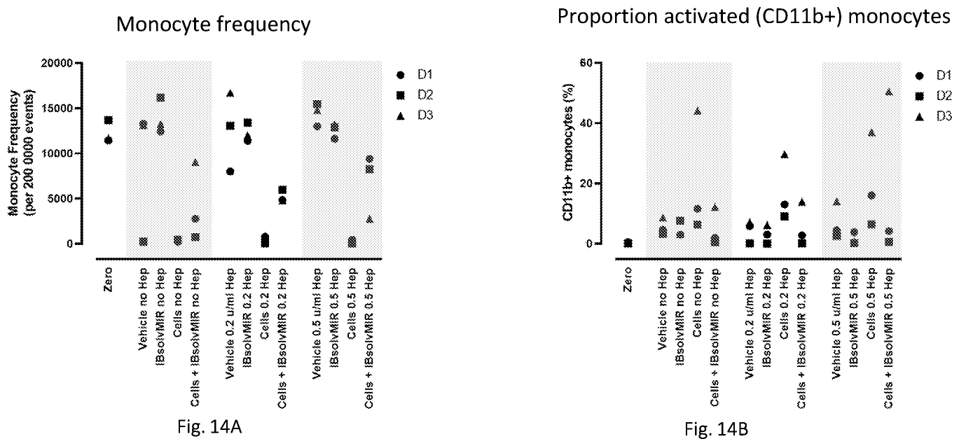

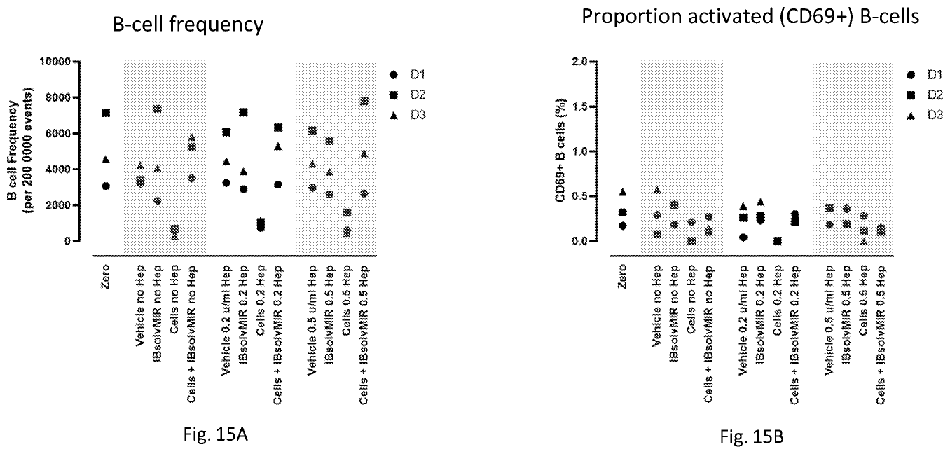

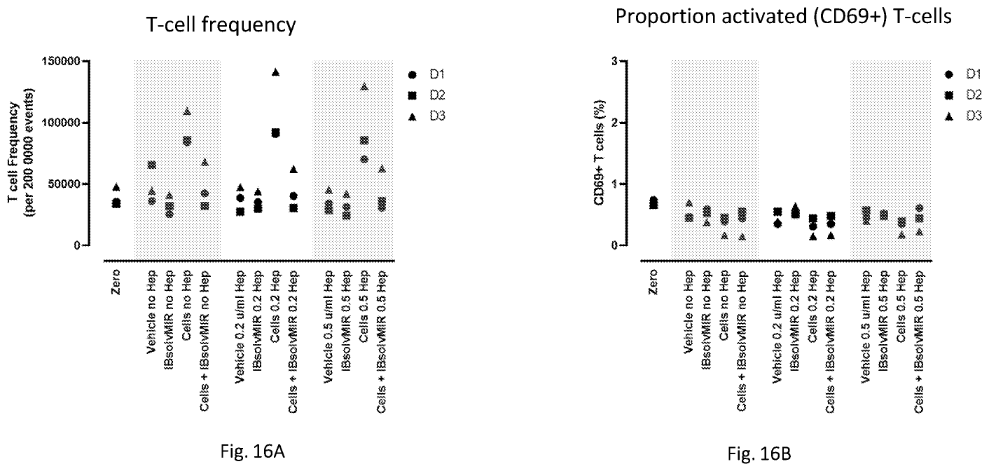

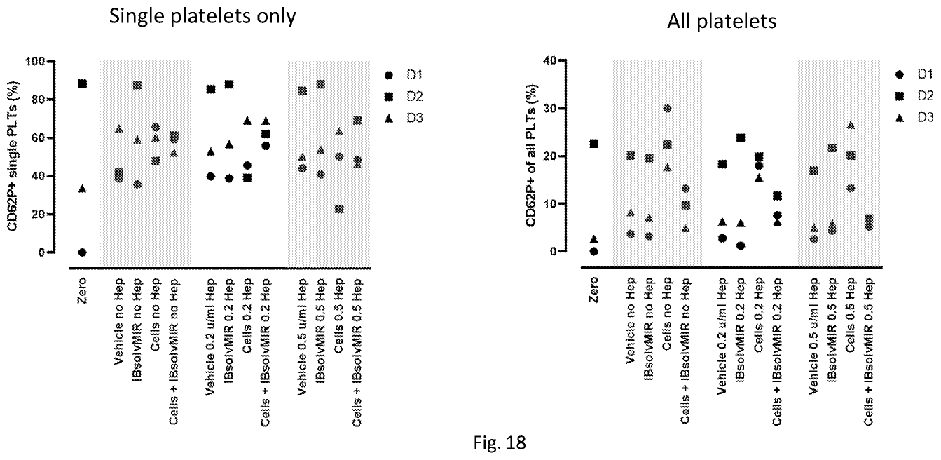

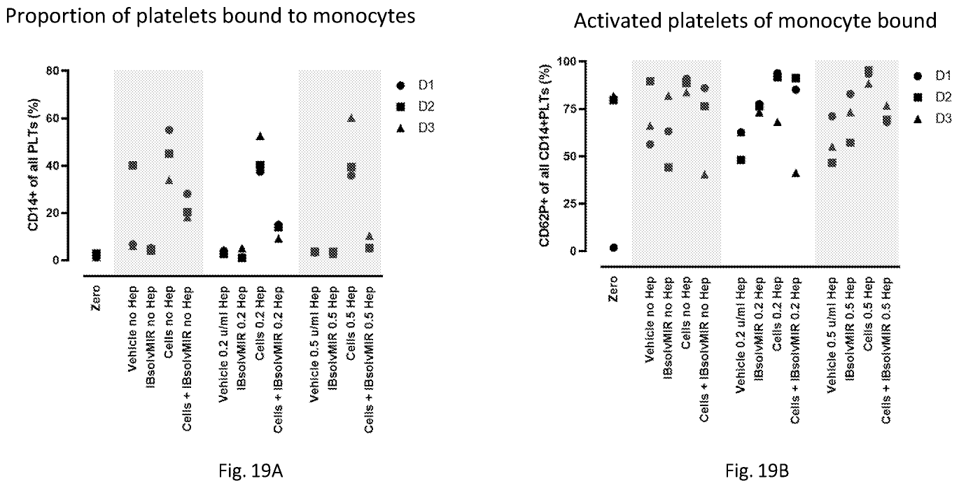

Granulocytes, monocytes and platelets were activated by addition of WI-38 cells along with decreased frequencies of these cell types (FIGS. 13A and 13B, 14A and 14B, 18). Co-administration of IBsolvMIR® overall decreased activation profile of monocytes (FIGS. 14A and 14B) and granulocytes (FIGS. 13A and 13B) and increased frequency of these cell types. T and B cells were not activated by addition of WI-38 cells (FIGS. 15A and 15B, 16A and 16B). A clear WI-38 dependent decrease in B cell count was observed. This decrease was entirely reversed up to vehicle level by co-administration of IBsolvMIR® (FIGS. 15A and 15B).

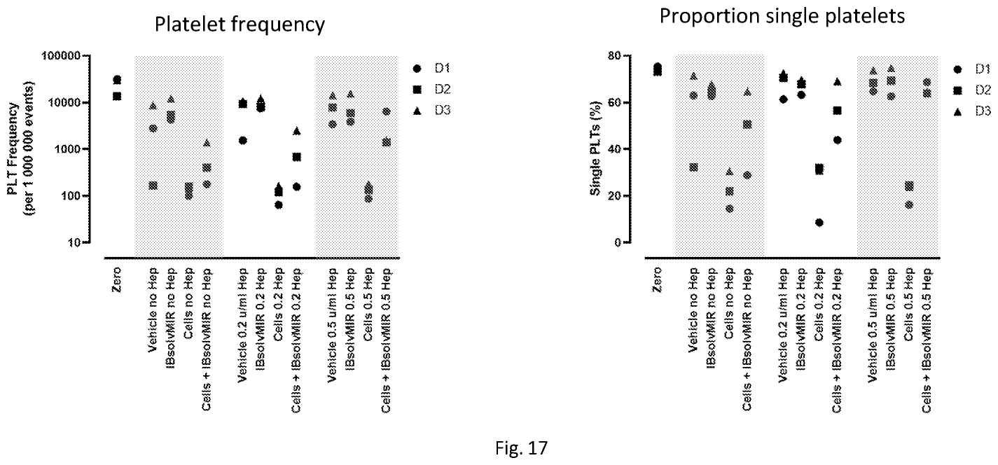

The decrease in platelet counts in response to WI-38 addition as monitored by cell counter was confirmed by flow cytometry (FIG. 17). This decrease was accompanied by an increased binding of platelets to granulocytes and monocytes (FIGS. 19A and 19B, 20A and 20B). WI-38 also triggered aggregation of platelets monitored as a decrease in proportion of single platelets, an effect entirely revoked by IBsolvMIR® (FIG. 17). Platelet binding to WI-38 cells was observed in all donors at all heparin concentrations and partly reverted by IBsolvMIR® (FIG. 21A).

Complement activation on platelets following WI-38 treatment was also observed as an increased C5b-9 formation on platelets, an effect partly reverted by IBsolvMIR® (FIG. 21B). Heparin potentiated the effects of IBsolvMIR® in suppressing platelet binding to WI-38 cells and on C5b-9 formation on platelets (FIGS. 21A and 21B).

Complement Analysis

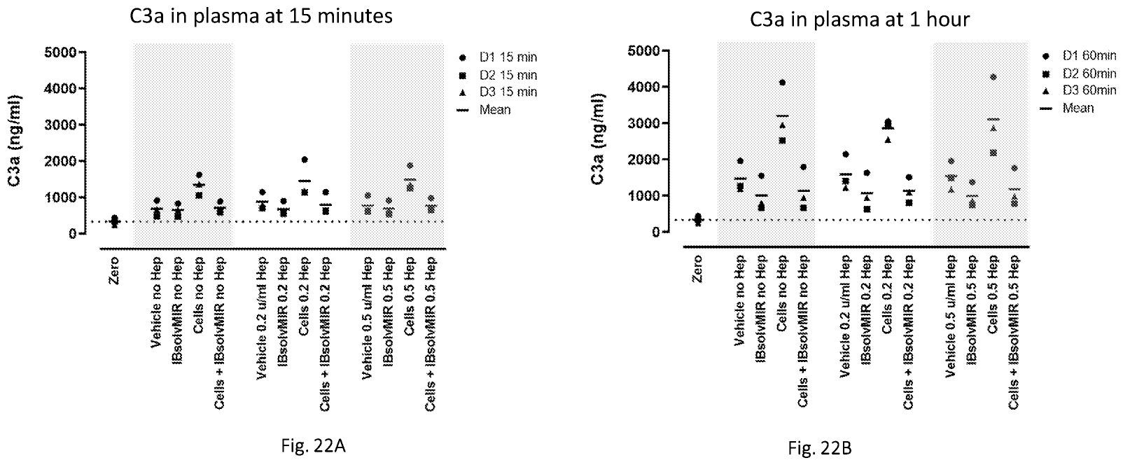

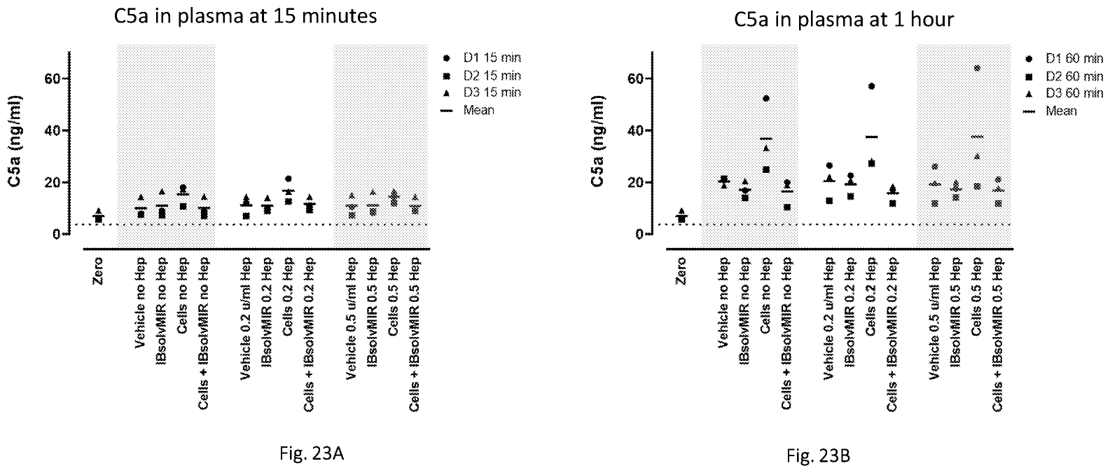

Complement split products C3a and C5a were studied by ELISA. C3a was increased in the WI-38 treated groups already at the 15 min time point by about two-fold (FIG. 22A). This increase was entirely blocked by IBsolvMIR® (FIG. 22A). At 1 hour, the vehicle groups were more clearly elevated over zero, and the WI-38 groups were further elevated but the relative change was still about two-fold (FIG. 22B). In all three main groups (no heparin, 0.2 and 0.5 U/ml heparin), IBsolvMIR® decreased C3a compared to corresponding treatment without IBsolvMIR® (vehicle vs IBsolvMIR® alone and WI-38 cells alone vs WI-38+IBsolvMIR®) (FIGS. 22A and 22B). The pattern was very similar for C5a as for C3a but the relative differences were smaller for C5a (FIGS. 23A and 23B). Yet, at the 1 hour time point, just as for C3a, C5a was lower in almost every donor in every group when comparing IBsolvMIR® treated vs corresponding treatment without IBsolvMIR® (FIG. 23B).

### CONCLUSION

WI-38 cells were viable and TF expression was nearly total in viable cell population. IBsolvMIR® was effective in the enhancement of TF expression in the presence of WI-38 cells. The effect of IBsolvMIR® was further manifested by restoring the number of WBCs, increased numbers of and decreased activation of granulocytes and monocytes as well as decreased platelet aggregation. Furthermore, complement activation on platelets and overall TF positive cells as well as monitored as C3a and C5a in plasma was inhibited by IBsolvMIR®.

Heparin potentiated the effects of IBsolvMIR® on complement activation on platelets and platelet binding to WI-38 cells. C5b-9 activation on platelets in the presence of IBsolvMIR® displayed a clear concentration-dependent effect by heparin not seen in the presence of WI-38 cells only.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1.-35. (canceled)

36. A method for treating or preventing thromboinflammation comprising administering a combination comprising an effective amount of dextran sulfate, or a pharmaceutically acceptable salt thereof, and an effective amount of heparin, or a pharmaceutically acceptable salt thereof, to a subject.

37. The method according to claim 36, wherein administering the combination comprises simultaneously administering the effective amount of dextran sulfate, or the pharmaceutically acceptable salt thereof, and the effective amount of heparin, or the pharmaceutically acceptable salt thereof to the subject.

38. The method according to claim 36, wherein administering the combination comprises sequentially administering the effective amount of dextran sulfate, or the pharmaceutically acceptable salt thereof, and the effective amount of heparin, or the pharmaceutically acceptable salt thereof to the subject.

39. The method according to claim 36, wherein the dextran sulfate, or the pharmaceutically acceptable salt thereof, has an average molecular weight equal to or below 10 000 Da.

40. The method according to claim 39, wherein the average molecular weight is within a range of from 3 000 to 10 000 Da.

41. The method according to claim 40, wherein the average molecular weight is within a range of from 3 500 to 9 500 Da.

42. The method according to claim 41, wherein the average molecular weight is within a range of from 4 500 to 7 500 Da.

43. The method according to claim 42, wherein the average molecular weight is within a range of from 4 500 to 5 500 Da.

44. The method according to claim 36, wherein the dextran sulfate, or the pharmaceutically acceptable salt thereof, has an average sulfur content in a range of from 15 to 20%.

45. The method according to claim 44, wherein the dextran sulfate, or the pharmaceutically acceptable salt thereof, has an average sulfur content of about 17%.

46. The method according to claim 36, wherein the dextran sulfate, or the pharmaceutically acceptable salt thereof, has a number average molecular weight (Mₙ) as measured by nuclear magnetic resonance (NMR) spectroscopy within a range of from 1850 to 3500 Da.

47. The method according to claim 46, wherein the dextran sulfate, or the pharmaceutically acceptable salt thereof, has a Mₙ as measured by NMR spectroscopy within a range of from 1850 to 2500 Da.

48. The method according to claim 47, wherein the dextran sulfate, or the pharmaceutically acceptable salt thereof, has a Mₙ as measured by NMR spectroscopy within a range of from 1850 to 2300 Da.

49. The method according to claim 48, wherein the dextran sulfate, or the pharmaceutically acceptable salt thereof, has a Mₙ as measured by NMR spectroscopy within a range of from 1850 to 2000 Da.

50. The method according to claim 46, wherein the dextran sulfate, or the pharmaceutically acceptable salt thereof, has an average sulfate number per glucose unit within a range of from 2.5 to 3.0.

51. The method according to claim 50, wherein the dextran sulfate, or the pharmaceutically acceptable salt thereof, has an average sulfate number per glucose unit within a range of from 2.5 to 2.8.

52. The method according to claim 51, wherein the dextran sulfate, or the pharmaceutically acceptable salt thereof, has an average sulfate number per glucose unit within a range of from 2.6 to 2.7.

53. The method according to claim 36, wherein the dextran sulfate, or the pharmaceutically acceptable salt thereof, has on average 5.1 glucose units and an average sulfate number per glucose unit of 2.6 to 2.7.

54. The method according to claim 36, wherein the pharmaceutically acceptable salt of dextran sulfate is a sodium salt of dextran sulfate.

55. The method according to claim 36, wherein the heparin, or the pharmaceutically acceptable salt thereof, has an average molecular weight equal to or below 40 000 Da.

56. The method according to claim 55, wherein the heparin, or the pharmaceutically acceptable salt thereof, has an average molecular weight equal to or below 30 000 Da.

57. The method according to claim 56, wherein the heparin, or the pharmaceutically acceptable salt thereof, has an average molecular weight equal to or below 20 000 Da.

58. The method according to claim 57, wherein the heparin, or the pharmaceutically acceptable salt thereof, has an average molecular weight equal to or below 17 500 Da.

59. The method according to claim 58, wherein the heparin, or the pharmaceutically acceptable salt thereof, has an average molecular weight equal to or below 10 000 Da.

60. The method according to claim 59, wherein the heparin, or the pharmaceutically acceptable salt thereof, has an average molecular weight selected within a range of from 2 000 to 10 000 Da.

61. The method according to claim 60, wherein the heparin, or the pharmaceutically acceptable salt thereof, has an average molecular weight selected within a range of from 3 000 to 10 000 Da.

62. The method according to claim 61, wherein the heparin, or the pharmaceutically acceptable salt thereof, has an average molecular weight selected within a range of from 3 500 to 9 500 Da.

63. The method according to claim 62, wherein the heparin, or the pharmaceutically acceptable salt thereof, has an average molecular weight selected within a range of from 3 500 to 5 500 Da.

64. The method according to claim 55, wherein the heparin, or the pharmaceutically acceptable salt thereof, is unfractionated heparin, or a pharmaceutically acceptable salt thereof, comprising anionic, sulfated glycosaminoglycan polymers with weights within an interval of from 1 000 Da to 40 000 Da.

65. The method according to claim 64, wherein the unfractionated heparin, or a pharmaceutically acceptable salt thereof, has an average molecular weight selected within an interval of from 2 000 Da to 35 000 Da.

66. The method according to claim 65, wherein the unfractionated heparin, or a pharmaceutically acceptable salt thereof, has an average molecular weight selected within an interval of from 3 000 and 30 000 Da.

67. The method according to claim 66, wherein the unfractionated heparin, or a pharmaceutically acceptable salt thereof, has an average molecular weight selected within an interval of from 10 000 Da to 20 000 Da.

68. The method according to claim 67, wherein the unfractionated heparin, or a pharmaceutically acceptable salt thereof, has an average molecular weight selected within an interval of from 10 000 Da to 17 500 Da.

69. The method according to claim 68, wherein the unfractionated heparin, or a pharmaceutically acceptable salt thereof, has an average molecular weight selected within an interval of from 12 000 Da to 15 000 Da.

70. The method according to claim 36, wherein the pharmaceutically acceptable salt of heparin is a sodium salt of heparin.

71. The method according to claim 36, wherein the thromboinflammation is selected from the group consisting of instant blood-mediated inflammatory reaction (IBMIR) and ischemia-reperfusion injury (IRI).

72. A transplant composition comprising a transplant, dextran sulfate, or a pharmaceutically acceptable salt thereof, and heparin, or a pharmaceutically acceptable salt thereof.

73. The transplant composition according to claim 72, wherein the transplant composition is a cell transplant composition comprising a cell transplant, dextran sulfate, or the pharmaceutically acceptable salt thereof, and heparin, or the pharmaceutically acceptable salt thereof, and a solvent or solution, which then comprises dextran sulfate, or the pharmaceutically acceptable salt thereof, and heparin, or the pharmaceutically acceptable salt thereof, and the cell transplant.

74. The transplant composition according to claim 72, wherein the transplant composition is an organ transplant composition comprising an organ or tissue transplant, dextran sulfate, or the pharmaceutically acceptable salt thereof, and heparin, or the pharmaceutically acceptable salt thereof, and an organ preservation solution, which then comprises dextran sulfate, or the pharmaceutically acceptable salt thereof, and heparin, or the pharmaceutically acceptable salt thereof, with the organ or tissue transplant immersed in the organ preservation solution.

75. An in vitro instant blood-mediated inflammatory reaction (IBMIR) model method comprising:
adding in vitro test cells to a blood loop comprising whole blood to trigger IBMIR;
adding in vitro dextran sulfate, or a pharmaceutically acceptable salt thereof, to the blood loop;
adding in vitro heparin, or a pharmaceutically acceptable salt thereof, to the whole human blood in the blood loop; and
determining an effectiveness of a combination of dextran sulfate, or the pharmaceutically acceptable salt thereof, and heparin, or the pharmaceutically acceptable salt thereof, to inhibit IBMIR by determining at least one hematological parameter, at least one coagulation parameter, at least one complement parameter and at least one platelet parameter.

76. The in vitro method according to claim 75, wherein the hematological parameter is selected from the group consisting of white blood cell (WBC) count, neutrophil count, monocyte count, eosinophil count and a combination thereof.

77. The in vitro method according to claim 75, wherein the coagulation parameter is selected from the group consisting of activated partial thromboplastin time (APTT), blood clot size, hemolysis and a combination thereof.

78. The in vitro method according to claim 75, wherein the complement parameter is selected from the group consisting of C3a, C5a and combination thereof.

79. The in vitro method according to claim 75, wherein the platelet parameter is selected from the group consisting of proportion of platelets bound to the test cells, proportion of platelets bound to monocytes, proportion of monocytes bond to granulocytes, proportion of C5b-9 positive platelets and a combination thereof.

80. An in vitro instant blood-mediated inflammatory reaction (IBMIR) model kit comprising:
a tubing;
a connector configured to interconnect the tubing to form a tubing loop;
dextran sulfate, or a pharmaceutically acceptable salt thereof; and
heparin, or a pharmaceutically acceptable salt thereof, wherein the heparin, or the pharmaceutically salt thereof, is not immobilized to an inner surface of the tubing or the connector.

81. The kit according to claim 80, further comprising test cells.

82. The kit according to claim 80, wherein
the tubing is a heparinized tubing; and
the connector is a heparinized connector.

83. The kit according to claim 80, further comprising whole blood.
